(19) United States
(12) Patent Application Publication
(10) Pub. No.: US 2026/0301131 A1
(43) Pub. Date: Oct. 1, 2026

(54) Generation Of Artificial Contrast-Enhanced Radiological Images

(71) Applicant: Bayer Aktiengesellschaft, Leverkusen (DE)

(72) Inventors: Matthias LENGA, Leverkusen (DE); Ivo Matteo BALTRUSCHAT, Leverkusen (DE); Parvaneh JANBAKHSHI, Leverkusen (DE); Felix Karl KREIS, Leverkusen (DE)

(21) Appl. No.: 19/490,162

(22) PCT Filed: May 30, 2024
(86) PCT No.: PCT/EP2024/064881

§ 371 Date: Dec. 4, 2025

(30) Foreign Application Priority Data
Jun. 5, 2023 (EP) 23177301.1
Jul. 13, 2023 (EP) 23185296.3

(51) Int. Cl. G06T 5/60; G06T 5/50; G06T 11/60

(52) U.S. Cl. CPC G06T 5/60; G06T 5/50; G06T 11/60; G06T 2207/10081; G06T 2207/10088; G06T 2207/10116; G06T 2207/20081; G06T 2207/20084; G06T 2207/20224; G06T 2207/30056; G06T 2207/30096; G06T 2210/41

(57) ABSTRACT

The present invention relates to the technical field of generating artificial contrast-enhanced radiological images.

### TECHNICAL FIELD

The present disclosure is concerned with the technical field of generation of artificial contrast-enhanced radiological images.

### INTRODUCTION

WO2019/074938A 1 discloses a method for reducing the amount of contrast agent in the generation of radiological images with the aid of an artificial neural network.

In the disclosed method, a training data set is created in a first step. The training data set comprises for each person of a multiplicity of persons i) a native radiological image (zero-contrast image), ii) a radiological image after administration of a small amount of contrast agent (low-contrast image) and iii) a radiological image after administration of a standard amount of contrast agent (full-contrast image). The standard amount is the amount recommended by the manufacturer and/or distributor of the contrast agent and/or the amount approved by a regulatory authority and/or the amount specified in a package leaflet for the contrast agent.

In a second step, an artificial neural network is trained to predict for each person of the training data set, on the basis of the native image and the image after administration of an amount of contrast agent smaller than the standard amount, an artificial radiological image showing an acquisition region after administration of the standard amount of contrast agent. The measured radiological image after administration of a standard amount of contrast agent serves in each case as reference (ground truth) in the training.

In a third step, the trained artificial neural network can be used to predict for a new person, on the basis of a native image and of a radiological image after administration of an amount of contrast agent smaller than the standard amount, an artificial radiological image showing the acquired region as it would look if a standard amount of contrast agent had been administered.

The method disclosed in WO2019/074938A 1 has disadvantages.

The artificial neural network disclosed in WO2019/074938A1 is trained to predict a radiological image after administration of the standard amount of a contrast agent. The artificial neural network is not configured and not trained to predict a radiological image after administration of an amount smaller or higher than the standard amount of contrast agent. The method described in WO2019/074938A 1 can in principle be trained to predict a radiological image after administration of an amount of contrast agent different than the standard amount, but this requires further training data and further training.

The medical images generated by trained machine-learning models may contain errors (see for example K. Schwarz et al.: On the Frequency Bias of Generative Models, https://doi.org/10.48550/arXiv.2111.02447).

Such errors (artefacts) can be problematic, since a doctor might make a diagnosis and/or initiate therapy on the basis of the artificial medical images. When reviewing artificial medical images, a doctor needs to know whether features in the artificial medical images are due to real features of the examination object or whether they are artefacts due to errors in prediction by the trained machine-learning model.

It would be desirable to be able to generate radiological images with variable contrast enhancement without needing to generate training data for each individual contrast enhancement and without needing to train an artificial neural network. It would additionally be desirable to be able to generate radiological images with variable contrast enhancement using a trackable deterministic process to generate the variable contrast enhancement. This facilitates the approval and use of a corresponding medical procedure, while minimizing false negative and false positive results. Machine learning methods employ statistical models that are of limited generalizability because they are usually based on a limited selection of training data. It would additionally be desirable to be able to generate radiological images with variable contrast enhancement using a wide variety of contrast agents. It would additionally be desirable to be able to use the method for generating radiological images with variable contrast enhancement using a wide variety of different contrast agents irrespective of their physical, chemical, physiological or other properties. It would additionally be desirable to be able to generate radiological images with variable contrast enhancement that have fewer errors (artefacts).

### SUMMARY

These and other objects are achieved by the subject matter of the independent claims. Preferred embodiments of the present disclosure are found in the dependent claims, in the present description and in the drawings.

The present disclosure thus provides in a first aspect a computer-implemented method for generating a synthetic contrast-enhanced radiological image, the method comprising the steps of:
providing a first representation, where the first representation represents an examination region of an examination object without contrast agent or after administration of a first amount of a contrast agent,
providing a second representation, where the second representation represents the examination region of the examination object after administration of a second amount of the contrast agent, the second amount being larger than the first amount,
feeding the first representation and the second representation to a first model, where the first model is configured to generate, based on the first representation and the second representation, a third representation, where the third representation represents the examination region of the examination object after administration of a third amount of the contrast agent, the third amount being different from the first amount and the second amount,
feeding the third representation to a second model,
   where the second model has been trained in a training process based on training data,
   where the training data for each reference object comprise a multiplicity of reference objects:
      (i) a reference representation generated by the first model that represents a reference region of the reference object after administration of a reference amount of the contrast agent, and
      (ii) a measured reference representation of the reference region of the reference object after administration of the reference amount of the contrast agent,
   where the training process for each reference object comprises the steps of:
      feeding the reference representation generated by the first model to the second model,
      receiving a corrected reference representation from the second model,
      reducing the differences between the corrected reference representation and the measured reference representation by modifying model parameters,
receiving a corrected third representation of the examination region of the examination object from the second model,
outputting and/or storing the corrected third representation and/or transmitting the corrected third representation to a separate computer system.

The present disclosure further provides a computer system comprising:
a processor; and
a storage medium that stores an application program configured to perform an operation when executed by the processor, said operation comprising the steps of
   providing a first representation, where the first representation represents an examination region of an examination object without contrast agent or after administration of a first amount of a contrast agent,
   providing a second representation, where the second representation represents the examination region of the examination object after administration of a second amount of the contrast agent, the second amount being larger than the first amount,
   feeding the first representation and the second representation to a first model, where the first model is configured to generate, based on the first representation and the second representation, a third representation, where the third representation represents the examination region of the examination object after administration of a third amount of the contrast agent, the third amount being different from the first amount and the second amount,
   feeding the third representation to a second model,
      where the second model has been trained in a training process based on training data,
      where the training data for each reference object comprise a multiplicity of reference objects:
         (i) a reference representation generated by the first model that represents a reference region of the reference object after administration of a reference amount of the contrast agent, and
         (ii) a measured reference representation of the reference region of the reference object after administration of the reference amount of the contrast agent,
      where the training process for each reference object comprises the steps of
         feeding the reference representation generated by the first model to the second model,
         receiving a corrected reference representation from the second model,
         reducing the differences between the corrected reference representation and the measured reference representation by modifying model parameters,
receiving a corrected third representation of the examination region of the examination object from the second model,
outputting and/or storing the corrected third representation and/or transmitting the corrected third representation to a separate computer system.

The present disclosure further provides a computer program that can be loaded into a working memory of a computer system, where it causes the computer system to execute the following steps:
providing a first representation, where the first representation represents an examination region of an examination object without contrast agent or after administration of a first amount of a contrast agent,
providing a second representation, where the second representation represents the examination region of the examination object after administration of a second amount of the contrast agent, the second amount being larger than the first amount,
feeding the first representation and the second representation to a first model, where the first model is configured to generate, based on the first representation and the second representation, a third representation, where the third representation represents the examination region of the examination object after administration of a third amount of the contrast agent, the third amount being different from the first amount and the second amount,
feeding the third representation to a second model,
   where the second model has been trained in a training process based on training data,
   where the training data for each reference object comprise a multiplicity of reference objects:
      (i) a reference representation generated by the first model that represents a reference region of the reference object after administration of a reference amount of the contrast agent, and
      (ii) a measured reference representation of the reference region of the reference object after administration of the reference amount of the contrast agent,
   where the training process for each reference object comprises the steps of
      feeding the reference representation generated by the first model to the second model,
      receiving a corrected reference representation from the second model,
      reducing the differences between the corrected reference representation and the measured reference representation by modifying model parameters,
receiving a corrected third representation of the examination region of the examination object from the second model,
outputting and/or storing the corrected third representation and/or transmitting the corrected third representation to a separate computer system.

The present disclosure further provides for the use of a contrast agent in a radiological examination method comprising the steps of
providing a first representation, where the first representation represents an examination region of an examination object without the contrast agent or after administration of a first amount of the contrast agent,
providing a second representation, where the second representation represents the examination region of the examination object after administration of a second amount of the contrast agent, the second amount being larger than the first amount,
feeding the first representation and the second representation to a first model, where the first model is configured to generate, based on the first representation and the second representation, a third representation, where the third representation represents the examination region of the examination object after administration of a third amount of the contrast agent, the third amount being different from the first amount and the second amount,
feeding the third representation to a second model,
   where the second model has been trained in a training process based on training data,
   where the training data for each reference object comprise a multiplicity of reference objects:
      (i) a reference representation generated by the first model that represents a reference region of the reference object after administration of a reference amount of the contrast agent, and
      (ii) a measured reference representation of the reference region of the reference object after administration of the reference amount of the contrast agent,
   where the training process for each reference object comprises the steps of
      feeding the reference representation generated by the first model to the second model,
      receiving a corrected reference representation from the second model,
      reducing the differences between the corrected reference representation and the measured reference representation by modifying model parameters,
receiving a corrected third representation of the examination region of the examination object from the second model,
outputting and/or storing the corrected third representation and/or transmitting the corrected third representation to a separate computer system.

The present disclosure further provides a contrast agent for use in a radiological examination method comprising the steps of
providing a first representation, where the first representation represents an examination region of an examination object without the contrast agent or after administration of a first amount of the contrast agent,
providing a second representation, where the second representation represents the examination region of the examination object after administration of a second amount of the contrast agent, the second amount being larger than the first amount,
feeding the first representation and the second representation to a first model, where the first model is configured to generate, based on the first representation and the second representation, a third representation, where the third representation represents the examination region of the examination object after administration of a third amount of the contrast agent, the third amount being different from the first amount and the second amount,
feeding the third representation to a second model,
   where the second model has been trained in a training process based on training data,
   where the training data for each reference object comprise a multiplicity of reference objects:
      (i) a reference representation generated by the first model that represents a reference region of the reference object after administration of a reference amount of the contrast agent, and
      (ii) a measured reference representation of the reference region of the reference object after administration of the reference amount of the contrast agent,
   where the training process for each reference object comprises the steps of:
      feeding the reference representation generated by the first model to the second model,
      receiving a corrected reference representation from the second model,
      reducing the differences between the corrected reference representation and the measured reference representation by modifying model parameters,
receiving a corrected third representation of the examination region of the examination object from the second model,
outputting and/or storing the corrected third representation and/or transmitting the corrected third representation to a separate computer system.

The present disclosure further provides a kit comprising a computer program product and a contrast agent, wherein the computer program product comprises a computer program that can be loaded into a working memory of a computer system, where it causes the computer system to execute the following steps:
providing a first representation, where the first representation represents an examination region of an examination object without the contrast agent or after administration of a first amount of the contrast agent,
providing a second representation, where the second representation represents the examination region of the examination object after administration of a second amount of the contrast agent, the second amount being larger than the first amount,
feeding the first representation and the second representation to a first model, where the first model is configured to generate, based on the first representation and the second representation, a third representation, where the third representation represents the examination region of the examination object after administration of a third amount of the contrast agent, the third amount being different from the first amount and the second amount,
feeding the third representation to a second model,
   where the second model has been trained in a training process based on training data,
   where the training data for each reference object comprise a multiplicity of reference objects:
      (i) a reference representation generated by the first model that represents a reference region of the reference object after administration of a reference amount of the contrast agent, and
      (ii) a measured reference representation of the reference region of the reference object after administration of the reference amount of the contrast agent,
   where the training process for each reference object comprises the steps of:
      feeding the reference representation generated by the first model to the second model,
      receiving a corrected reference representation from the second model,
      reducing the differences between the corrected reference representation and the measured reference representation by modifying model parameters,
receiving a corrected third representation of the examination region of the examination object from the second model,
outputting and/or storing the corrected third representation and/or transmitting the corrected third representation to a separate computer system.

### BRIEF DESCRIPTION OF THE DRAWINGS

FIG. 1 shows by way of example and in schematic form the generation of a third representation of an examination region of an examination object on the basis of a first representation and a second representation with the aid of a first model.

FIG. 2 shows schematically a further example of the generation of a third representation of an examination region of an examination object on the basis of a first and a second representation of the examination region of the examination object with the aid of a first model.

FIG. 3 shows by way of example and in schematic form a process for training a second model.

FIG. 4 shows by way of example and in schematic form the generation of a corrected third representation based on a third representation with the aid of a trained second model.

FIG. 5 shows by way of example and in schematic form a model comprising a first model and a second model.

FIG. 6 shows by way of example and in schematic form a computer system according to the present disclosure.

FIG. 7 shows by way of example and in schematic form a further embodiment of the computer system according to the present disclosure.

FIG. 8 shows by way of example and in schematic form one embodiment of the computer-implemented method of the present disclosure in the form of a flowchart.

### DETAILED DESCRIPTION

The subject matters of the present disclosure will be more particularly elucidated below, without distinguishing between the subject matters (method, computer system, computer program (product), use, contrast agent for use, kit). Rather, the elucidations that follow are intended to apply by analogy to all subject matters, irrespective of the context (method, computer system, computer program (product), use, contrast agent for use, kit) in which they occur.

Where steps are stated in an order in the present description or in the claims, this does not necessarily mean that this disclosure is limited to the order stated. Instead, it is conceivable that the steps are also executed in a different order or else in parallel with one another, the exception being when one step builds on another step, thereby making it imperative that the step building on the previous step be executed next (which will however become clear in the individual case). The stated orders thus constitute preferred embodiments.

In certain places the invention will be more particularly elucidated with reference to drawings. The drawings show specific embodiments having specific features and combinations of features, which are intended primarily for illustrative purposes; the invention is not to be understood as being limited to the features and combinations of features shown in the drawings. Furthermore, statements made in the description of the drawings in relation to features and combinations of features are intended to be generally applicable, that is to say transferable to other embodiments too and not limited to the embodiments shown.

The present disclosure describes means by which one or more artificial radiological images are generated on the basis of at least two representations representing an examination region of an examination object after addition/administration/use of varying amounts of contrast agent, in which the contrast between regions with contrast agent and regions without contrast agent can be varied.

The “examination object” is normally a living being, preferably a mammal, most preferably a human.

The “examination region” is a part of the examination object, for example an organ or part of an organ or a plurality of organs or another part of the examination object.

For example, the examination region may be a liver, kidney, heart, lung, brain, stomach, bladder, prostate, intestine or a part of said parts or another part of the body of a mammal (for example a human).

In one embodiment, the examination region includes a liver or part of a liver or the examination region is a liver or part of a liver of a mammal, preferably a human.

In a further embodiment, the examination region includes a brain or part of a brain or the examination region is a brain or part of a brain of a mammal, preferably a human.

In a further embodiment, the examination region includes a heart or part of a heart or the examination region is a heart or part of a heart of a mammal, preferably a human.

In a further embodiment, the examination region includes a thorax or part of a thorax or the examination region is a thorax or part of a thorax of a mammal, preferably a human.

In a further embodiment, the examination region includes a stomach or part of a stomach or the examination region is a stomach or part of a stomach of a mammal, preferably a human.

In a further embodiment, the examination region includes a pancreas or part of a pancreas or the examination region is a pancreas or part of a pancreas of a mammal, preferably a human.

In a further embodiment, the examination region includes a kidney or part of a kidney or the examination region is a kidney or part of a kidney of a mammal, preferably a human.

In a further embodiment, the examination region includes one or both lungs or part of a lung of mammal, preferably a human.

In a further embodiment, the examination region includes a breast or part of a breast or the examination region is a breast or part of a breast of a female mammal, preferably a female human.

In a further embodiment, the examination region includes a prostate or part of a prostate or the examination region is a prostate or part of a prostate of a male mammal, preferably a male human.

The examination region, also referred to as the field of view (FOV), is in particular a volume that is imaged in radiological images. The examination region is typically defined by a radiologist, for example on a localizer image. It is of course also possible for the examination region to be alternatively or additionally defined in an automated manner, for example on the basis of a selected protocol.

The examination region is subjected to a radiological examination.

“Radiology” is the branch of medicine that is concerned with the use of electromagnetic rays and mechanical waves (including for instance ultrasound diagnostics) for diagnostic, therapeutic and/or scientific purposes. Besides X-rays, other ionizing radiation such as gamma radiation or electrons are also used. Imaging being a key application, other imaging methods such as sonography and magnetic resonance imaging (nuclear magnetic resonance imaging) are also counted as radiology, even though no ionizing radiation is used in these methods. The term “radiology” in the context of the present disclosure thus encompasses in particular the following examination methods: computed tomography, magnetic resonance imaging, sonography.

In one embodiment of the present disclosure, the radiological examination is a magnetic resonance imaging examination.

In a further embodiment, the radiological examination is a computed tomography examination.

In a further embodiment, the radiological examination is an ultrasound examination.

In radiological examinations, contrast agents are commonly used for contrast enhancement.

“Contrast agents” are substances or mixtures of substances that improve the depiction of structures and functions of the body in radiological examinations.

In computed tomography, iodine-containing solutions are normally used as contrast agents. In magnetic resonance imaging (MRI), superparamagnetic substances (for example iron oxide nanoparticles, superparamagnetic iron-platinum particles (SIPPs)) or paramagnetic substances (for example gadolinium chelates, manganese chelates, hafnium chelates) are normally used as contrast agents. In the case of sonography, liquids containing gas-filled microbubbles are normally administered intravenously. Examples of contrast agents can be found in the literature (see for example A. S. L. Jascinth et al.: Contrast Agents in computed tomography: A Review, Journal of Applied Dental and Medical Sciences, 2016, vol. 2, issue 2, 143-149; H. Lusic et al.: X-ray-Computed Tomography Contrast Agents, Chem. Rev. 2013, 113, 3, 1641-1666; https://www.radiology.wisc.edu/wp-content/uploads/2017/10/contrast-agents-tutorial.pdf, M. R. Nouh et al.: Radiographic and magnetic resonances contrast agents: Essentials and tipsfor safe practices, World J Radiol. 2017 Sep. 28; 9(9): 339-349; L. C. Abonyi et al.: Intravascular Contrast Media in Radiography: Historical Development & Review of Risk Factors for Adverse Reactions, South American Journal of Clinical Research, 2016, vol. 3, issue 1, 1-10; ACR Manual on Contrast Media, 2020, ISBN: 978-1-55903-012-0; A. Ignee et al.: Ultrasound contrast agents, Endosc Ultrasound. 2016 November-December; 5(6): 355-362).

MRI contrast agents exert their effect in an MRI examination by altering the relaxation times of structures that take up contrast agents. A distinction can be made between two groups of substances: paramagnetic and superparamagnetic substances. Both groups of substances have unpaired electrons that induce a magnetic field around the individual atoms or molecules. Superparamagnetic contrast agents result in a predominant shortening of T2, whereas paramagnetic contrast agents mainly result in a shortening of T1. The effect of said contrast agents is indirect, since the contrast agent does not itself emit a signal, but instead merely influences the intensity of signals in its vicinity. An example of a superparamagnetic contrast agent is iron oxide nanoparticles (SPIO, superparamagnetic iron oxide). Examples of paramagnetic contrast agents are gadolinium chelates such as gadopentetate dimeglumine (trade name: Magnevist® and others), gadoteric acid (Dotarem®, Dotagita®, Cyclolux®), gadodiamide (Omniscan®), gadoteridol (ProHance®), gadobutrol (Gadovist®), gadopiclenol (Elucirem, Vueway) and gadoxetic acid (Primovist®/Eovist®).

In one embodiment, the radiological examination is an MRI examination in which an MRI contrast agent is used.

In a further embodiment, the radiological examination is a CT examination in which a CT contrast agent is used.

In a further embodiment, the radiological examination is a CT examination in which an MRI contrast agent is used.

The generation of an artificial radiological image with variable contrast enhancement is based on at least two representations of the examination region, a first representation and a second representation.

The first representation represents the examination region without contrast agent or after administration of a first amount of a contrast agent. Preferably, the first representation represents the examination region without contrast agent (native representation).

The second representation represents the examination region after administration of a second amount of a contrast agent. The second amount is larger than the first amount (it being possible also for the first amount to be zero, as described). The expression “after administration of a second amount of a contrast agent” should not be understood as meaning that the first amount and the second amount in the examination region are added together. Thus, the expression “the representation represents the examination region after administration of a (first or second) amount” should rather be understood as meaning: “the representation represents the examination region with a (first or second) amount” or “the representation represents the examination region including a (first or second) amount”.

In one embodiment, both the first amount and the second amount of the contrast agent are smaller than the standard amount.

In a further embodiment, the second amount of the contrast agent corresponds to the standard amount.

In a further embodiment, the first amount of the contrast agent is equal to zero and the second amount of the contrast agent is smaller than the standard amount.

In a further embodiment, the first amount of the contrast agent is equal to zero and the second amount of the contrast agent corresponds to the standard amount.

The standard amount is normally the amount recommended by the manufacturer and/or distributor of the contrast agent and/or approved by a regulatory authority and/or the amount specified in a package leaflet for the contrast agent.

For example, the standard amount of Primovist® is 0.025 mmol Gd-EOB-DTPA disodium/kg body weight.

In one embodiment of the present disclosure, the contrast agent is an agent that includes gadolinium(III) 2-[4,7,10-tris(carboxymethyl)-1,4,7,10-tetrazacyclododec-1-yl]acetic acid (also referred to as gadolinium-DOTA or gadoteric acid).

In a further embodiment, the contrast agent is an agent that includes gadolinium(III) ethoxybenzyldiethylenetriaminepentaacetic acid (Gd-EOB-DTPA); preferably, the contrast agent includes the disodium salt of gadolinium(III) ethoxybenzyldiethylenetriaminepentaacetic acid (also referred to as gadoxetic acid).

In one embodiment of the present disclosure, the contrast agent is an agent that includes gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate (also referred to as gadopiclenol) (see for example WO2007/042504 and WO2020/030618 and/or WO2022/013454).

In one embodiment of the present disclosure, the contrast agent is an agent that includes dihydrogen [(±)-4-carboxy-5,8,11-tris(carboxymethyl)-1-phenyl-2-oxa-5,8,11-triazatridecan-13-oato(5-)]gadolinate(2-) (also referred to as gadobenic acid).

In one embodiment of the present disclosure, the contrast agent is an agent that includes tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate (also referred to as gadoquatrane) (see for example J. Lohrke et al.: Preclinical Profile of Gadoquatrane: A Novel Tetrameric, Macrocyclic High Relaxivity Gadolinium-Based Contrast Agent. Invest Radiol., 2022, 1, 57(10): 629-638; WO2016193190).

In one embodiment of the present disclosure, the contrast agent is an agent that comprises a Gd³⁺ complex of a compound of the formula (I)

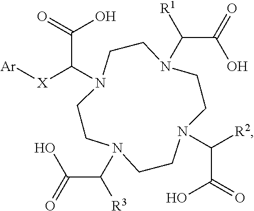

where
Ar is a group selected from

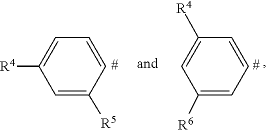

where _{#} is the linkage to X,
X is a group selected from
CH₂, (CH₂)₂, (CH₂)₃, (CH₂)₄ and *—(CH₂)₂O—CH₂—^{#},
where * is the linkage to Ar and ^{#} is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof.

In one embodiment of the present disclosure, the contrast agent is an agent that comprises a Gd³⁺ complex of a compound of the formula (II)

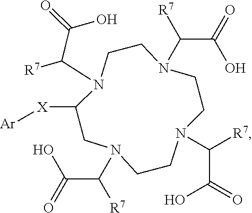

where
Ar is a group selected from

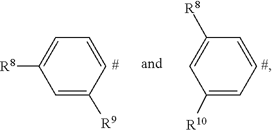

where _{#} is the linkage to X,
X is a group selected from CH₂, (CH₂)₂, (CH₂)₃, (CH₂)₄ and *—(CH₂)₂O—CH₂—^{#}, where * is the linkage to Ar and ^{#} is the linkage to the acetic acid residue,
R⁷ is a hydrogen atom or a group selected from C₁-C₃ alkyl, —CH₂OH, —(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

The term “C₁-C₃ alkyl” denotes a linear or branched, saturated monovalent hydrocarbon group having 1, 2 or 3 carbon atoms, for example methyl, ethyl, n-propyl or isopropyl. The term “C₂-C₄ alkyl” denotes a linear or branched, saturated monovalent hydrocarbon group having 2, 3 or 4 carbon atoms.

The term “C₂-C₄ alkoxy” refers to a linear or branched, saturated monovalent group of the formula (C₂-C₄ alkyl)-O—, in which the term “C₂-C₄ alkyl” is as defined above, for example a methoxy, ethoxy, n-propoxy or isopropoxy group.

In one embodiment of the present disclosure, the contrast agent is an agent that includes gadolinium 2,2′,2″-(10-{1-carboxy-2-[2-(4-ethoxyphenyl)ethoxy]ethyl}-1,4,7,10-tetraazacyclododecane-1,4,7-triyl)triacetate (see for example WO2022/194777, example 1).

In one embodiment of the present disclosure, the contrast agent is an agent that includes gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 2).

In one embodiment of the present disclosure, the contrast agent is an agent that includes gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 4).

In one embodiment of the present disclosure, the contrast agent is an agent that includes gadolinium (2S,2'S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate (see for example WO2022/194777, example 15).

In one embodiment of the present disclosure, the contrast agent is an agent that includes gadolinium 2,2′,2″-{10-[(1S)-4-(4-butoxyphenyl)-1-carboxybutyl]-1,4,7,10-tetraazacyclododecane-1,4,7-triyl}triacetate (see for example WO2022/194777, example 31).

In one embodiment of the present disclosure, the contrast agent is an agent that includes gadolinium 2,2′,2″-{(2S)-10-(carboxymethyl)-2-[4-(2-ethoxyethoxy)benzyl]-1,4,7,10-tetraazacyclododecane-1,4,7-triyl}triacetate.

In one embodiment of the present disclosure, the contrast agent is an agent that includes gadolinium 2,2′,2″-[10-(carboxymethyl)-2-(4-ethoxybenzyl)-1,4,7,10-tetraazacyclododecane-1,4,7-triyl]triacetate.

In one embodiment of the present disclosure, the contrast agent is an agent that includes gadolinium(III) 5,8-bis(carboxylatomethyl)-2-[2-(methylamino)-2-oxoethyl]-10-oxo-2,5,8,11-tetraazadodecane-1-carboxylate hydrate (also referred to as gadodiamide).

In one embodiment of the present disclosure, the contrast agent is an agent that includes gadolinium(III) 2-[4-(2-hydroxypropyl)-7,10-bis(2-oxido-2-oxoethyl)-1,4,7,10-tetrazacyclododec-1-yl]acetate (also referred to as gadoteridol).

In one embodiment of the present disclosure, the contrast agent is an agent that includes gadolinium(III) 2,2′,2″-(10-((2R,3S)-1,3,4-trihydroxybutan-2-yl)-1,4,7,10-tetraazacyclododecane-1,4,7-triyl)triacetate (also referred to as gadobutrol or Gd-DO3A-butrol).

In a first step, the first representation and the second representation are provided, i.e. received or generated, for example.

The term “receiving” encompasses both the retrieving of representations and the accepting of representations transmitted for example to the computer system of the present disclosure. The representations may be received from a computed tomography system, from a magnetic resonance imaging system or from an ultrasound scanner. The radiological images may be read from one or more data storage media and/or transmitted from a separate computer system.

The term “generating” preferably means that a representation is generated on the basis of another (for example a received) representation or on the basis of a plurality of other (for example received) representations. For example, a received representation may be a representation of an examination region of an examination object in real space. On the basis of this real-space representation it is possible for example to generate a representation of the examination region of the examination object in frequency space through a transform operation (for example a Fourier transform). Further options for generating a representation based on one or more other representations are described in this description.

A representation of the examination region may for the purposes of the present disclosure be a representation in real space (image space), a representation in frequency space, a representation in projection space or a representation in another space.

In a representation in real space, also referred to in this description as real-space depiction or real-space representation, the examination region is normally represented by a large number of image elements (for example pixels or voxels or doxels), which may for example be in a raster arrangement in which each image element represents a part of the examination region, wherein each image element may be assigned a colour value or grey value. The colour value or grey value represents a signal intensity, for example the attenuation of X-rays. A format widely used in radiology for storing and processing representations in real space is the DICOM format. DICOM (Digital Imaging and Communications in Medicine) is an open standard for storing and exchanging information in medical image data management.

In a representation in frequency space, also referred to in this description as frequency-space depiction or frequency-space representation, the examination region is represented by a superposition of fundamental oscillations. For example, the examination region may be represented by a sum of sine and cosine functions having different amplitudes, frequencies and phases. The amplitudes and phases may be plotted as a function of the frequencies, for example, in a two- or three-dimensional representation. Normally, the lowest frequency (origin) is placed in the centre. The further away from this centre, the higher the frequencies. Each frequency can be assigned an amplitude, representing the frequency in the frequency-space depiction, and a phase indicating the extent of the shift of the respective oscillation with respect to a sine or cosine oscillation.

A representation in real space can for example be converted (transformed) by a Fourier transform into a representation in frequency space. Conversely, a representation in frequency space can for example be converted (transformed) by an inverse Fourier transform into a representation in real space.

Details about real-space depictions and frequency-space depictions and their respective interconversion are described in numerous publications, see for example https:/see.stanford.edu/materials/lsoftaee261/book-fall-07.pdf.

A representation of an examination region in projection space is normally the result of a computed tomography examination prior to image reconstruction. A projection-space depiction can be understood as meaning raw data in the computed tomography examination. In computed tomography, the intensity or attenuation of X-radiation as it passes through the examination object is measured. From this, projection values can be calculated. In a second step, the object information encoded by the projection is transformed into an image (real-space depiction) through a computer-aided reconstruction. The reconstruction can be effected with the Radon transform. The Radon transform describes the link between the unknown examination object and its associated projections.

Details about the transformation of projection data into a real-space depiction are described in numerous publications, see for example K. Fang: The Radon Transformation and Its Application in Tomography, Journal of Physics Conference Series 1903(1):012066.

A representation of the examination region can also be a representation in Hough space. For the recognition of geometric objects in an image, edge detection is followed by the creation, by what is known as a Hough transform, of a dual space in which all possible parameters of the geometric object are entered for each point in the image lying at an edge. Each point in dual space accordingly corresponds to a geometric object in image space. For a straight line this can be for example the slope and the y-intercept of the straight line and for a circle this can be the centre and radius of the circle. Details about the Hough transform can be found in the literature (see for example A. S. Hassanein et al.: A Survey on Hough Transform, Theory, Techniques and Applications, arXiv: 1502.02160v1).

There are further spaces in which it is possible for there to be representations of the examination region.

For the sake of simplicity and clarity, the invention is in large parts of the description described on the basis of real-space representations. This should not however be understood as limiting. Those skilled in the art of image analysis know how to apply the appropriate parts of the description to representations other than real-space representations.

The first representation and the second representation are fed to a first model.

It is possible to co-register the first representation and the second representation before feeding them to the first model. “Co-registration” (also known in the prior art as “image registration”) is employed to bring two or more real-space depictions of the same examination region into the best possible alignment with one another. One of the real-space depictions is defined as the reference image, the other is termed the object image. In order to optimally fit this to the reference image, a compensating transformation is calculated.

It is also possible to co-register representations in frequency space; it should be noted here that a translation in real space constitutes an additive linear phase ramp in frequency space. Scaling and rotation are on the other hand retained in the Fourier and inverse Fourier transform—scaling and rotation in frequency space is also scaling and rotation in real space (see for example S. Skare: Rigid Body Image Realignment in Image Space vs. k-Space, ISMRM Scientific Workshop on Motion Correction, 2014, https://cds.ismrm.org/protected/Motion_14/Program/Syllabus/Skare.pdf).

The first model is configured to generate, based on the first representation and the second representation, a third representation. The third representation represents the examination region of the examination object after administration of a third amount of the contrast agent.

The third amount of the contrast agent is different from the first amount and the second amount. The third amount of the contrast agent is preferably larger than the second amount, but the third amount may also be smaller than the second amount.

For example, if the second amount is smaller than the standard amount, the third amount may be equal to the standard amount. However, it is also possible for the third amount to be larger than the standard amount.

If the third amount is smaller than the second amount, the first model will result in an attenuation in contrast. This means that the contrast enhancement produced by the contrast agent in the second representation is attenuated (less pronounced) in the third representation.

If the third amount is larger than the second amount, the first model will result in contrast enhancement. This means that the contrast enhancement produced by the contrast agent in the second representation is enhanced (more pronounced) in the third representation.

The first model may be a machine-learning model.

A “machine learning model” can be understood as meaning a computer-implemented data processing architecture. Such a model is able to receive input data and to supply output data on the basis of said input data and model parameters. Such a model is able to learn a relationship between the input data and the output data through training. During training, the model parameters can be adjusted so as to supply a desired output for a particular input.

During the training of such a model, the model is presented with training data from which it can learn. The trained machine-learning model is the result of the training process. Besides input data, the training data include the correct output data (target data) that the model is intended to generate based on the input data. During training, patterns that map the input data onto the target data are identified.

In the training process, the input data of the training data are input into the model, and the model generates output data. The output data are compared with the target data. Model parameters are altered so as to reduce the differences between the output data and the target data to a (defined) minimum. The modification of model parameters in order to reduce the differences can be done using an optimization method such as a gradient descent method.

The differences can be quantified with the aid of a loss function. A loss function of this kind can be used to calculate a loss value for a given pair of output data and target data. The aim of the training process may consist of altering (adjusting) the parameters of the machine-learning model so as to reduce the loss value for all pairs of the training data set to a (defined) minimum.

For example, if the output data and the target data are numbers, the loss function can be the absolute difference between these numbers. In this case, a high absolute loss value can mean that one or more model parameters need to be altered to a substantial degree.

For example, for output data in the form of vectors, difference metrics between vectors such as the mean squared error, a cosine distance, a norm of the difference vector such as a Euclidean distance, a Chebyshev distance, an Lp norm of a difference vector, a weighted norm or any other type of difference metric between two vectors can be chosen as the loss function.

In the case of higher-dimensional outputs, such as two-dimensional, three-dimensional or higher-dimensional outputs, an element-by-element difference metric can for example be used. Alternatively or in addition, the output data may be transformed into for example a one-dimensional vector before calculation of a loss value.

The first model may be a machine-learning model, as described for example in one of the following publications: WO2019/074938A1, WO2022/253687A1, WO2022/207443A1, WO2022/223383A1, WO20227184297A1, WO2022/179896A2, WO2021/069338A1, EP 22209510.1, EP23159288.2, PCT/EP2023/053324, PCT/EP2023/050207, CN110852993A, CN110853738A, US2021150671A1, arXiv:2303.15938v1, doi:10.1093/jrr/rrz030.

The first model may be a mechanistic (deterministic) model. Mechanistic models are based on fundamental principles and known relationships within a system. They are often derived from scientific theories and area-specific knowledge. Mechanistic models describe the underlying mechanisms of a system using mathematical equations or physical laws. They aim to simulate the behaviour ofthe system based on an understanding of its components and interactions.

Machine learning models are on the other hand data-driven and learn patterns and relationships from input data without explicitly programming the relationships.

Thus, whereas mechanistic models are based on fundamental principles and aim to represent the underlying system mechanisms, machine learning models learn patterns and relationships directly from data without explicitly programming these relationships.

The mechanistic model is thus based on physical laws. In radiological examinations, a signal produced by a contrast agent is normally dependent on the amount (e.g. concentration) of the contrast agent in the examination region. For example, the signal strength may over a defined concentration range show a linear dependence or another form of dependence on the concentration of the contrast agent in the examination region. The functional dependence of the signal strength on the concentration can be utilized to create a mechanistic model.

In one embodiment, the first model is a mechanistic model that in a first step determines, based on the first representation and the second representation, the signal intensity distribution produced by the contrast agent in the examination region and in a second step adds this a times to the first or second representation, α being a gain factor.

The generation of the signal intensity distribution produced by the contrast agent in the examination region may for example include a subtraction of the first representation from the second representation. When the first representation represents the examination region of the examination object without contrast agent and the second representation represents the examination region of the examination object with contrast agent, subtracting the first representation from the second representation will generate a representation of the examination region in which the signal intensity distribution is produced by the contrast agent alone, since the signals that are not produced by the contrast agent will be the same in the first representation and the second representation and will be eliminated by the subtraction.

If this signal intensity distribution is added once (α=1) to the first representation, the second representation is again obtained.

If this signal intensity distribution is added multiple times (α>1) to the first representation, a third representation of the examination region is obtained in which the contrast between regions with contrast agent and regions without contrast agent is enhanced relative to the second representation. It is possible to have not just integer values for α, but other real values (for example 1.5 or 3.1416 or other values) too.

If a fraction (0>α>1) of this signal intensity distribution is added to the first representation, a third representation of the examination region is obtained in which the contrast between regions with contrast agent and regions without contrast agent is attenuated relative to the second representation.

Negative α values are also possible, which can for example be selected so that regions of the investigation region that experience a contrast agent-induced signal enhancement in the representation generated by measurement are completely dark (black) in the artificially generated representations.

The gain factor α is thus a positive or negative real number. The gain factor α may be chosen by a user, i.e. it may be variable or predefined, i.e. predetermined. The gain factor α may also be determined automatically, for example based on the histogram of the first representation and/or of the second representation and/or on the difference of the first representation from the second representation and/or with the aid of an initial model (see below).

Varying the gain factor α thus allows the contrast between regions with contrast agent and regions without contrast agent to be varied.

Thus, with the aid of the first model it is possible, based on the first representation and the second representation, to generate a third representation of the examination region of the examination object that represents the examination region after administration of a third amount, it being possible for the third amount to be different from the first amount and the second amount.

With the aid of the first model it is possible, based on a first representation of the examination region without contrast agent or with a first amount of a contrast agent, and on a second representation of the examination region with a second amount of the contrast agent that is less than or equal to the standard amount, to generate a representation of the examination region with an amount of contrast agent that is larger than the standard amount.

The mechanistic model described above is based on the assumption that the signal intensity represented by grey values or colour values in a representation of the examination region shows linear dependence on the amount of contrast agent administered. This is the case particularly in many MRI examinations.

The linear dependence allows the contrast to be varied by varying the gain factor α. A gain factor of α=2 thus means that the third amount of contrast agent is equal to twice the second amount.

The mechanistic model described above is disclosed in EP22207079.9, EP22207080.7 and EP23168725.2.

It should be noted that instead of linear dependence, the mechanistic model can also be based on another dependence. The dependence can be determined empirically.

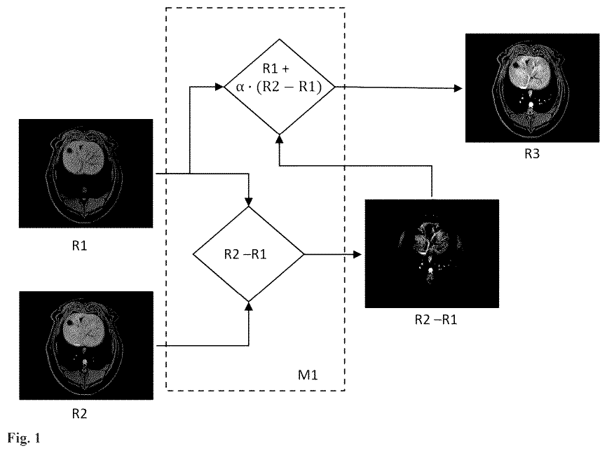

FIG. 1 shows by way of example and in schematic form the generation of a third representation of an examination region of an examination object on the basis of a first representation and a second representation with the aid of a first model.

The examination object is a pig and the examination region includes the pig's liver.

The first representation R1 is a magnetic resonance image that represents the examination region in real space without contrast agent.

The second representation R2 represents the same examination region of the same examination object as the first representation R1 in real space. The second representation R2 is likewise a magnetic resonance image.

The second representation R2 represents the examination region after administration of a second amount of a contrast agent. In the present example, an amount of 25 μmol per kg body weight of a hepatobiliary contrast agent was administered intravenously to the examination object. The second representation R2 represents the examination region in the so-called arterial phase (see for example DOI:10.1002/jmri.22200).

A hepatobiliary contrast agent has the characteristic features of being specifically taken up by liver cells (hepatocytes), accumulating in the functional tissue (parenchyma) and enhancing contrast in healthy liver tissue. An example of a hepatobiliary contrast agent is the disodium salt of gadoxetic acid (Gd-EOB-DTPA disodium), which is described in U.S. Pat. No. 6,039,931A and is commercially available under the trade names Primovist® and Eovist®. Further hepatobiliary contrast agents are described inter alia in WO2022/194777.

The first representation R1 and the second representation R2 are fed to a first model M1. On the basis of the first representation R1 and the second representation R2, the first model M1 generates a third representation R3. In the example shown in FIG. 1, the generation of the third representation R3 involves subtracting the first representation R1 from the second representation R2 (R2−R1). The generation of the third representation R3 additionally involves adding the difference of the first representation from the second representation to the first representation a times (R3=R1+α·(R2−R1)).

If negative grey/colour values occur when subtracting the first representation R1 from the second representation R2, these negative values can be set to zero (or another value) to avoid such negative values.

The difference (R2−R1) represents the contrast enhancement (signal intensity distribution) produced in the examination region by the second amount of the contrast agent.

The difference (R2−R1) is multiplied by the gain factor α and the multiplication result added to the first representation R1. This generates the third representation R3. In the example shown in FIG. 1, the gain factor α=3, i.e. the difference (R2−R1) is added to the first representation R1 three times.

The third representation R3 can be subjected to a normalization, that is to say the grey/colour values can be multiplied by a factor such that the grey/colour value having the highest value is represented for example by the grey tone/hue “white” and the grey/colour value having the lowest value is represented for example by the grey/colour tone “black”.

In the example shown in FIG. 1, the first model M1 thus consists of mathematical operations that execute the subtractions, multiplications and additions based on the grey/colour values of the individual image elements (for example pixels, voxels).

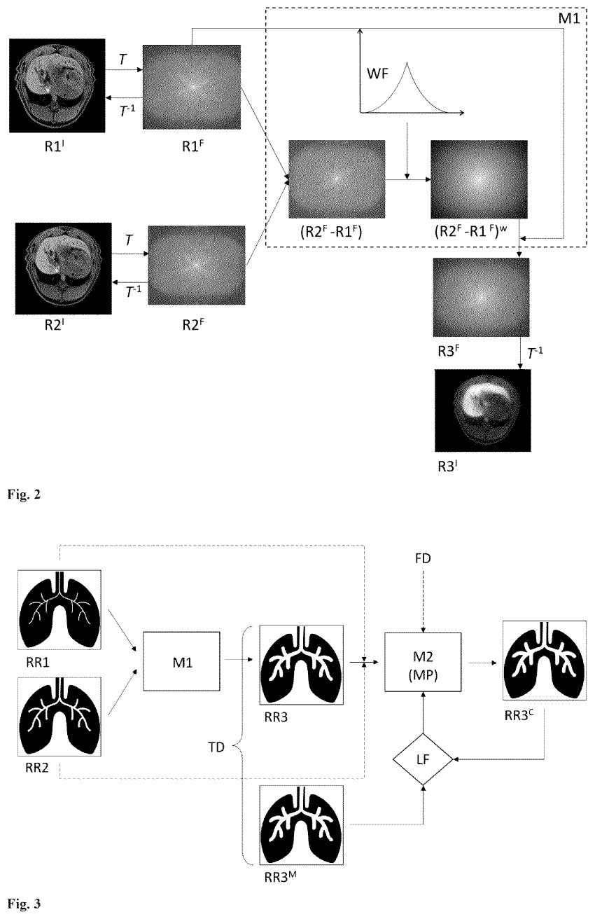

FIG. 2 shows schematically a further example of the generation of a third representation of an examination region of an examination object on the basis of a first and a second representation of the examination region of the examination object with the aid of a first model.

FIG. 2 shows an examination region of an examination object in the form of various representations.

A first representation R1^{I} represents the examination region in real space without contrast agent or after administration of a first amount of a contrast agent. The examination region shown in FIG. 2 includes a liver of a pig. The first representation R1^{I} is a magnetic resonance image.

The first real-space representation R1^{I} can be converted into a first representation R1^{F} of the examination region in frequency space through a transform operation T, for example a Fourier transform. The first frequency-space representation R1^{F} represents the same examination region of the same examination object as the first real-space representation R1^{I}, likewise without contrast agent or after administration of the first amount of the contrast agent.

The first frequency-space representation R1^{F} can be converted into the first real-space representation R1^{I} through a transform operation T⁻¹, for example by means of an inverse Fourier transform. The transform operation T⁻¹ is the inverse transform of transform operation T.

A second representation R2^{I} represents the same examination region of the same examination object as the first representation R1^{I} in real space. The second real-space representation R2^{I} represents the examination region after administration of a second amount of the contrast agent. The second amount is larger than the first amount (it being possible also for the first amount to be zero, as described). The second representation R2^{I} is likewise a magnetic resonance image. As contrast agent, the disodium salt of gadoxetic acid (Gd-EOB-DTPA disodium) was in the example shown in FIG. 2 used as a hepatobiliary MRI contrast agent.

The second real-space representation R2^{I} can be converted into a second representation R2^{F} of the examination region in frequency space by means of the transform operation T. The second frequency-space representation R2^{F} represents the same examination region of the same examination object as the second real-space representation R2^{I}, likewise after administration of the second amount of the contrast agent.

The second frequency-space representation R2^{F} can be converted into the second real-space representation R2^{I} by means of the transform operation T⁻¹.

In the example shown in FIG. 2, the first frequency-space representation R1^{F} and the second frequency-space representation R2^{F} are fed to a first model M1. On the basis of the first frequency-space representation R1^{F} and the second frequency-space representation R2^{F}, the first model M1 generates a third frequency-space representation R3^{F}. The third frequency-space representation R3^{F} can be converted into a third real-space representation R3^{I} through a transform operation T⁻¹ (for example an inverse Fourier transform).

The model M1 shown in FIG. 2 does not include the transform operation T that converts the first real-space representation R1^{I} into the first frequency-space representation R1^{F} and converts the second real-space representation R2^{I} into the second frequency-space representation R2^{F}. Likewise, the model M1 shown in FIG. 2 does not include the transform operation T⁻¹ that converts the third frequency-space representation R3^{F} into the third real-space representation R3^{I}. However, it is conceivable that the transform operation T and/or transform operation T⁻¹ are component(s) of the first model M1, i.e. it is conceivable that the first model M1 executes the transform operation T and/or transform operation T⁻¹.

The first model M1 subtracts the first frequency-space representation R1^{F} from the second frequency-space representation R2^{F} (R^{F}−R1^{F}). The result is a representation of the signal intensity distribution in frequency space produced by the contrast agent in the examination region.

The difference R2^{F}−R1^{F} is multiplied by a weight function WF that weights low frequencies more highly than high frequencies. In this case, the amplitudes of the fundamental oscillations are multiplied by a weight factor that increases as the frequencies become smaller. This step is an optional step that can be executed to increase the signal-to-noise ratio in the third representation, especially at higher values for the gain factor α (for example values greater than 3, 4, or 5). The result of this frequency-dependent weighting is the weighted representation (R2^{F}−R1^{F})^{W}.

Contrast information is represented in a frequency-space depiction by low frequencies, while the higher frequencies represent information about fine structures. Such weighting thus means that a higher weighting will be given to frequencies making a higher contribution to contrast than to those making a smaller contribution. Image noise is typically evenly distributed in the frequency depiction. The frequency-dependent weight function has the effect of a filter. The filter increases the signal-to-noise ratio by reducing the spectral noise density for high frequencies.

Preferred weight functions are Hann function (also referred to as the Hann window) and Poisson function (Poisson window).

Examples of other weight functions can be found for example at https://de.wikipedia.org/wiki/Fensterfunktion #Beispiele von_Fensterfunktionen; F. J. Harris et al.: On the Use of Windows for Harmonic Analysis with the Discrete Fourier Transform, Proceedings of the IEEE, vol. 66, No. 1, 1978; https://docs.scipy.org/doc/scipy/reference/signal.windows.html; K. M. M. Prabhu: Window Functions and Their Applications in Signal Processing, CRC Press, 2014, 978-1-4665-1583-3.

The weighted difference (R2^{F}−R1^{F})^{W} is in a next step multiplied by a gain factor α and added to the first frequency-space representation R1^{F}. The result is a third representation R3^{F}=R1^{F}+α·(R^{F}−R1^{F})^{W} of the examination region of the examination object in frequency space. The third frequency-space representation R3^{F} is in a further step converted into a third representation R3^{I} of the examination region of the examination object in real space by means of the transform operation T⁻¹ (for example an inverse Fourier transform).

The third representation R3^{I} represents the examination region of the examination object after administration of a third amount of the contrast agent. The third amount depends on the gain factor α. For example, if the gain factor is 3 and if the signal intensity distribution represented by the grey/colour values shows linear dependence on the amount of the contrast agent, then the third amount corresponds to three times the difference of the first amount from the second amount.

If a first model M1 as depicted in FIG. 1 is used to generate the third representation, then not only is the contrast enhanced by a gain factor greater than 1 (α>1), but the noise too is enhanced to the same extent. A first model M1 as depicted in FIG. 2 is able to achieve a certain reduction in noise through weighting with the weight function in frequency space.

In order to (further) reduce or eliminate noise and/or other unwanted artefacts in the third representation, the first model is followed by a second model. The third representation generated by the first model is thus not the result of the generation of the synthetic contrast-enhanced representation of the present disclosure. The second model serves for correction of the third representation generated by the first model. The term “correction” can in this instance mean reducing or eliminating noise and/or artefacts.

Thus, according to the present disclosure, two models are used for generating a synthetic contrast-enhanced radiological image: a first model and a second model. The first model serves for contrast enhancement (α>1) or contrast reduction (α<1). The second model serves for correction (for example noise suppression, artefact suppression). The first model can also be referred to as a synthesis model and the second model as a correction model.

In other words, according to the present disclosure two models are used to generate a synthetic contrast-enhanced radiological image: a first model and a second model. The first model generates a “suggestion” for a synthetic contrast-enhanced radiological image; the second model optimizes this “suggestion”. The “suggestion” can be a first approximation of the synthetic contrast-enhanced radiological image. The second model is able to modify (optimize) this approximation so that the result corresponds to a real contrast-enhanced radiological image.

The third representation generated by the first model is fed to the second model as input data and, based on this input data and based on model parameters, the second model generates a corrected third representation. In addition to the third representations generated by the first model, further data can be fed to the second model as input data (see below).

The second model is a machine-learning model. The second model has been trained on the basis of training data to generate, based on a third representation generated by the first model and on model parameters, a corrected (for example modified and/or optimized) third representation.

The training data include for each reference object of a multiplicity of reference objects: (i) a reference representation generated by the first model that represents a reference region of the reference object after administration of a reference amount of the contrast agent and (ii) a measured reference representation of the reference region of the reference region of the reference object after administration of the reference amount of the contrast agent.

The term “multiplicity” means more than ten, preferably more than one hundred.

The term “reference” is used in this description to distinguish the phase of training the second model from the phase of using the trained second model for the correction of a representation. The term “reference” otherwise has no limitation on meaning. The term “(reference) representation” means that the corresponding statement applies both to a representation of an examination object and to a reference representation of a reference object. A “reference object” is an object from which data (for example reference representations) are used to train the second model. On the other hand, data of an examination object are utilized in order to use the trained second model (in combination with the first model) for prediction. Each reference object is, like the examination object, normally a living being, preferably a mammal, most preferably a human. The “reference region” is a part of the reference object. The reference region is normally (but not necessarily) the examination region of the examination object. In other words, when the examination region is an organ or part of an organ (for example the liver or part of the liver) of the examination object, the reference region of each such reference object is preferably the corresponding organ or corresponding part of the organ of the respective reference object. The “reference amount” is an amount of contrast agent that is determined (defined) at least in part by the first model (for example, by the gain factor α). The “reference amount” may correspond to the third amount of contrast agent, but the “reference amount” may also be different from the third amount.

The training data with which the second model has been trained thus include (i) input data and (ii) target data. The second model is configured to generate output data on the basis of input data and model parameters. The output data are compared with the target data. Differences between the output data and the target data can be reduced by modifying model parameters in an optimization method (for example a gradient descent method).

For each reference object of the multiplicity of reference objects, the input data include a reference representation generated by the first model that represents the reference region of the reference object after administration of the reference amount of the contrast agent. The input data are thus generated with the aid of the first model. They are normally generated on the basis of a first reference representation and a second reference representation. The first reference representation represents the reference region of the respective reference object without contrast agent or after administration of a first reference amount of contrast agent. The first reference amount may correspond to the first amount. The second reference representation represents the reference region of the respective reference object after administration of a second reference amount of contrast agent. The second reference amount is normally larger than the first reference amount. The second reference amount may correspond to the second amount. The first reference representation and the second reference representation are fed to the first model and the first model generates a third reference representation. The third reference representation represents the reference region of the respective reference object after administration of a third reference amount of the contrast agent. The third reference amount is normally larger than the second reference amount. The third reference amount may correspond to the third amount. The third reference representation is the reference representation that is fed to the second model when training the second model.

The training data additionally include for each reference object a measured reference representation as target data. The measured reference representation represents the reference region of the respective reference object after administration of the third reference amount of the contrast agent. The measured reference representation is a measured representation; it thus represents the reference region of the respective reference object as it actually is after administration of the third reference amount of the contrast agent (ground truth) and, as the reference representation generated by the first model, should represent the reference region.

The training of the second model includes for each reference object of the multiplicity of reference objects:
feeding the (third) reference representation generated by the first model to the second model,
receiving a corrected reference representation from the second model,
reducing differences between the corrected reference representation and the measured reference representation by modifying model parameters.

The training process can be performed for as long as it takes for the differences to attain a predefined minimum and/or until the differences cannot be reduced further by modifying model parameters.

The second model is thus trained to correct the reference representations generated by the first model for different reference objects so as to approximate as closely as possible to the respective measured reference representation.

The second model is thus, for example, trained to reduce or eliminate noise and/or artefacts generated by the first model in the reference representations.

FIG. 3 shows by way of example and in schematic form a process for training a second model. The second model M2 is trained using training data TD. The training data TD include for each reference object of a multiplicity of reference objects, a third reference representation RR3 of a reference region of the respective reference object generated by a first model M1 and a measured third reference representation RR3^{M} of the reference region of the reference object. The training data may optionally additionally include further data (see below).

In the example shown in FIG. 3, only one set of training data TD for a single reference object is shown.

The reference object is a human; the reference region includes the lung of the human.

The measured third reference representation RR3^{M} represents the reference region of the reference object after administration of a third amount of a contrast agent. The measured third reference representation RR3^{M} is an image generated by measurement, that is to say the measured third reference representation RR3^{M} is the result of a radiological examination. For example, the measured third reference representation RR3^{M} may be a CT image, an MRI image, an ultrasound image, or another radiological image.

The third reference representation RR3 is generated by means of the first model M1. The first model M1 is configured to generate the third reference representation RR3 based on a first reference representation RR1 and a second reference representation RR2.

The first reference representation RR1 represents the reference region of the reference object without contrast agent or after administration of a first amount of the contrast agent. The second reference representation RR2 represents the reference region of the reference object after administration of a second amount of a contrast agent. The second amount is larger than the first amount. The third representation RR3 represents the reference region of the reference object after administration of the third amount of the contrast agent.

The third reference representation RR3 generated by the first model M1 is fed to the second model M2. It is optionally possible to additionally feed further input data to the second model, for example the first reference representation RR1 and/or the second reference representation RR2 and/or further/other data FD. The further input data may be data specifying the contrast agent used, the first amount of the contrast agent, the second amount of the contrast agent, the third amount of the contrast agent, the gain factor, acquisition parameters for generating the first and/or second representation, the reference object, the reference region and/or other properties/conditions. The optional use of further input data is symbolized in FIG. 3 by the dashed arrows. If further input data are used, these additional input data are likewise training data.

The second model M2 is configured to generate, based on the third reference representation RR3 and based on model parameters MP (and optionally further input data), a corrected third representation RR3^{C}. The second model M2 is trained to generate a corrected third representation RR3^{C} that approximates as closely as possible to the measured third representation RR3^{M}. For this, the corrected third representation RR3^{C} is compared with the measured third representation RR3^{M}. A loss function LF is used to quantify differences between the corrected third representation RR3^{C} and the measured third representation RR3^{M}. In an optimization method (for example a gradient descent method), model parameters MP are modified so as to reduce (minimize) the differences and thus the calculated loss determined using the loss function LF. The process is repeated for further training data sets of further reference objects until the differences have been reduced to a predefined minimum and/or the differences cannot be reduced further by modifying model parameters.

The trained second model can be stored in a data storage medium, transmitted to a separate computer system (for example over a network) and/or used to generate a corrected third representation of an examination region of an examination object.

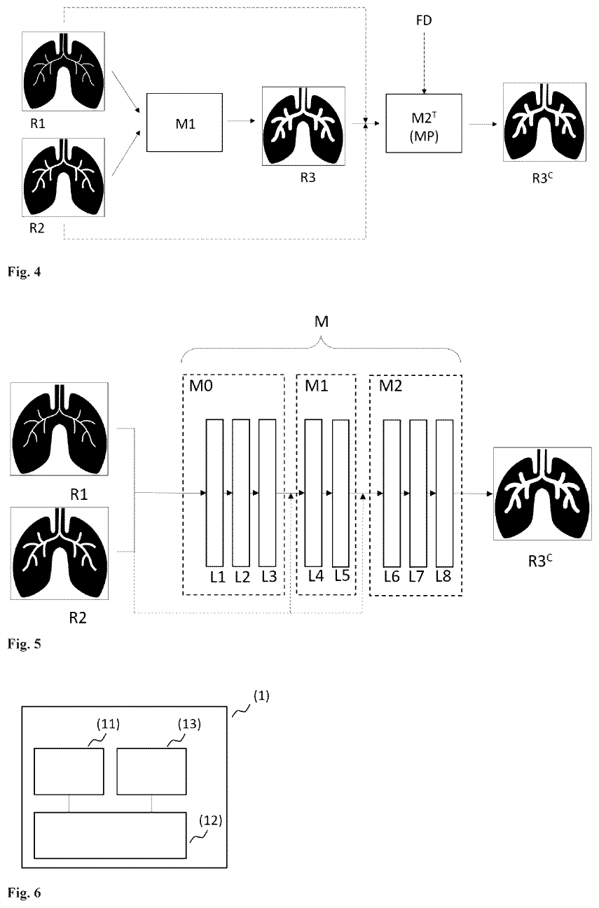

FIG. 4 shows by way of example and in schematic form the generation of a corrected third representation based on a third representation with the aid of a trained second model.

The trained second model M2^{T} may for example have been trained as described in relation to FIG. 3. The trained second model M2^{T} is fed with a third representation R3 of an examination region of an examination object. Further input data can optionally be fed to the trained second model M2^{T}. The further input data may be data specifying the contrast agent used, the first amount of the contrast agent, the second amount of the contrast agent, the third amount of the contrast agent, the gain factor, acquisition parameters for generating the first and/or second representation, the examination object, the examination region and/or other properties/conditions. The optional use of further input data is symbolized in FIG. 4 by the dashed arrows. Further input data are used in particular when such data have also been used to train the second model.

In the example shown in FIG. 4, the examination object is a human; the examination region includes the lung of the human.

The third representation R3 is generated with the aid of a first model M1. The first model M1 is configured to generate the third representation based on a first representation R1 and a second representation R2. The first representation R1 represents the examination region of the examination object without contrast agent or after administration of a first amount of a contrast agent. The second representation R2 represents the examination region of the examination object after administration of a second amount of the contrast agent. The second amount is larger than the first amount. The third representation R3 represents the examination region of the examination object after administration of a third amount of a contrast agent. The third amount is different from the first amount and the second amount. In the present example, the third amount is larger than the second amount, i.e. the first model generates a third representation R3 that exhibits contrast enhancement relative to the second representation R2.

The trained second model M2^{T} is configured and trained to generate, based on the third representation R3 generated by the first model M1 (and optionally further input data), a corrected third representation R3^{C}. The corrected third representation R3^{C} is an artificial radiological image that is improved relative to the third representation R3, since it contains, for example, less noise and/or fewer artefacts.

The second model may be an artificial neural network or include such a network.

An “artificial neural network” comprises at least three layers of processing elements: a first layer having input neurons (nodes), an N-th layer having at least one output neuron (node) and N−2 inner layers, where N is a natural number and greater than 2.

The input neurons serve to receive the input representations. Normally, there is one input neuron for each pixel or voxel of an input representation when the representation is a real-space depiction in the form of a raster graphic, or one input neuron for each frequency present in the input representation when the representation is a frequency-space depiction. There may be additional input neurons for additional input values (for example information about the examination region, about the examination object, about the conditions prevailing during the generation of the input representation, information about the state that the input representation represents, and/or information about the time or time interval at/during which the input representation had been generated).

The output neurons serve to output a synthetic radiological image.

The processing elements of the layers between the input neurons and the output neurons are connected to one another in a predetermined pattern with predetermined connection weights.

The artificial neural network may be a convolutional neural network (CNN for short) or include such a network.

A convolutional neural network is capable of processing input data in the form of a matrix. This makes it possible to use as input data digital radiological images depicted in the form of a matrix (e.g. width×height×colour channels). A normal neural network, for example in the form of a multilayer perceptron (MLP), requires on the other hand a vector as input, i.e. in order to use a radiological image as input, the pixels or voxels of the radiological image would have to be rolled out in a long chain one after the other. This means that normal neural networks are for example not able to recognize objects in a radiological image independently of the position of the object in the image. The same object at a different position in the image would have a completely different input vector.

A CNN normally consists essentially of an alternately repeating array of filters (convolutional layer) and aggregation layers (pooling layer) terminating in one or more layers of “normal” fully connected neurons (dense/fully connected layer).

The artificial neural network may have an autoencoder architecture, for example the artificial neural network may have an architecture such as the U-Net (see for example O. Ronneberger et al.: U-net: Convolutional networks for biomedical image segmentation, International Conference on Medical image computing and computer-assisted intervention, pages 234-241, Springer, 2015, https://doi.org/10.1007/978-3-319-24574-4_28).

The artificial neural network may be a generative adversarial network (GAN) (see for example M.-Y. Liu et al.: Generative Adversarial Networks for Image and Video Synthesis: Algorithms and Applications, arXiv:2008.02793; J. Henry et al.: Pix2Pix GAN for Image-to-Image Translation, DOI: 10.13140/RG.2.2.32286.66887).

The artificial neural network may in particular be a generative adversarial network (GAN) for image super-resolution (SR) (see for example C. Ledig et al.: Photo-Realistic Single Image Super-Resolution Using a Generative Adversarial Network, arXiv: 1609.04802v5).

The artificial neural network may be a transformer network (see for example D. Karimi et al.: Convolution-Free Medical Image Segmentation using Transformers, arXiv:2102.13645 [eess.IV]).

If the first model is differentiable, then the first model and the second model can also be combined into a common model. In this case, the first model and the second model are components of a common model, where the third (reference) representation generated by the first model is fed directly to the second model.

The first model described in this description, for example in relation to FIGS. 1 and 2, is differentiable. It may for example as one or more layers prepending a second model designed as a neural network. Said one or more layers may include the calculation operations with the corresponding calculation parameters (e.g. the gain factor α) in the form of fixed (unchangeable) values, whereas model parameters of the second model may be variable. This allows the first model to be included in the training of the second model, with the model parameters of the first model remaining unchanged during the training. In such a case, the first model uses the unchangeable model parameters to specify the physical fundamentals that the second model must observe/accept/permit. In contrast to a fully variable model for generating artificial contrast-enhanced radiological images, as described for example in WO2019/074938A1, the approach described herein has the advantage that the physical fundamentals within which the model is able to move can be specified to the model as knowledge in the form of the first model. This makes the synthetic radiological images generated more realistic.

In a common model designed as a neural network comprising the first model and the second model, it is also possible to prepend one or more further layers to the first model. Said one or more further layers may for example include trainable (changeable) model parameters that provide for (improved) co-registration of the first (reference) representation and of the second (reference) representation. The (improved) co-registration can then be a component of the training. The one or more further layers prepending the first model may also include trainable model parameters that learn one or more model parameters of the first model, for example an optimized gain factor α and/or parameters of the weight function in the case of weighting as shown in FIG. 2. Said one or more layers prepending the first model may for example have an architecture such as a DenseNet (see for example G. Haung et al.: Densely Connected Convolutional Networks, arXiv:1608.06993v5).

A model comprising the first model and the second model can be trained in an end-to-end process.

It is additionally possible to feed the second model not just with the (reference) representation generated by the first model, but also with the first (reference) representation and/or the second (reference) representation. This allows the second model to be trained to reduce or eliminate artefacts in the third (reference) representation due to inadequate co-registration.

FIG. 5 shows by way of example and in schematic form a model comprising a first model and a second model.

The model M depicted in FIG. 5 has various processing layers L1, L2, L3, L4, L5, L6, L7 and L8. The number of processing layers was chosen purely at random; the processing layers depicted are for illustrative purposes only. Processing layers L4 and L5 form the first model M1; processing layers L6, L7 and L8 form the second model M2. The first model M1 is prepended by the processing layers L1, L2 and L3; these form an initial model M0.

To the initial model M0 are fed a first representation R1 and a second representation R2. The first representation R1 represents an examination region of an examination object without contrast agent or after administration of a first amount of a contrast agent; the second representation R2 represents the examination region of the examination object after administration of a second amount of the contrast agent; the second amount is larger than the first amount; the examination object is a human; the examination region includes the lung of the human.

The initial model M0 may be configured to co-register the first representation R1 and the second representation R2 and/or to determine model parameters of the first model M1. The co-registered representations can be forwarded via layer L3 of the initial M0 model to layer L4 of the first model M1. It is likewise possible at this point for model parameters determined by the initial model M0 to be forwarded to the first model M1, whereas the first representation R1 and the second representation R2 are fed to the first model M1 separately (see dashed arrow).

The first model M1 is configured to generate, based on the (co-registered) representation R1 and the (co-registered) representation R2, a third representation (not depicted in FIG. 5). The third representation represents the examination region of the examination object after administration of a third amount of the contrast agent; the third amount is different from the first amount and the second amount; preferably the third amount is larger than the second amount.

The third representation generated by the first model M1 is forwarded via layer L5 of the first model M1 to layer L6 of the second model M2.

The second model M2 can also be fed with the optionally co-registered first representation R1 and/or the optionally co-registered second representation R2 (see dashed arrow).

The second model M2 is configured and trained to generate, based on the third representation (and optionally based on the (co-registered) first representation R1 and/or (co-registered) second representation), a corrected third representation R3c. The corrected third representation R3c has less noise and/or fewer artefacts compared to the third representation. The corrected third representation R3c can be output via layer L8 of the second model M2.

FIG. 6 shows by way of example and in schematic form a computer system according to the present disclosure.

A “computer system” is an electronic data processing system that processes data by means of programmable calculation rules. Such a system typically comprises a “computer”, which is the unit that includes a processor for carrying out logic operations, and peripherals.

In computer technology, “peripherals” refers to all devices that are connected to the computer and are used for control of the computer and/or as input and output devices. Examples thereof are monitor (screen), printer, scanner, mouse, keyboard, drives, camera, microphone, speakers, etc. Internal ports and expansion cards are also regarded as peripherals in computer technology.

The computer system (1) shown in FIG. 6 comprises a receiving unit (11), a control and calculation unit (12) and an output unit (13).

The control and calculation unit (12) serves for control of the computer system (1), coordination of the data flows between the units of the computer system (1), and for the performance of calculations.

The control and calculation unit (12) is configured:
to generate a first representation or cause the receiving unit (11) to receive the first representation, where the first representation represents an examination region of an examination object without contrast agent or after administration of a first amount of a contrast agent,
to generate a second representation or to cause the receiving unit (11) to receive the second representation, where the second representation represents the examination region of the examination object after administration of a second amount of the contrast agent, the second amount being larger than the first amount,
to feed the first representation and the second representation to a first model, where the first model is configured to generate, based on the first representation and the second representation, a third representation, where the third representation represents the examination region of the examination object after administration of a third amount of the contrast agent, the third amount being different from the first amount and the second amount,
to feed the third representation to a second model,
   where the second model has been trained in a training process based on training data,
   where the training data for each reference object comprise a multiplicity of reference objects:
      (i) a reference representation generated by the first model that represents a reference region of the reference object after administration of a reference amount of the contrast agent, and
      (ii) a measured reference representation of the reference region of the reference object after administration of the reference amount of the contrast agent,
   where the training process for each reference object comprises the steps of:
      feeding the reference representation generated by the first model to the second model,
      receiving a corrected reference representation from the second model,
      reducing the differences between the corrected reference representation and the measured reference representation by modifying model parameters,
to receive a corrected third representation of the examination region of the examination object from the second model,
to cause the output unit (13) to output the corrected third representation, store it and/or transmit it to a separate computer system.

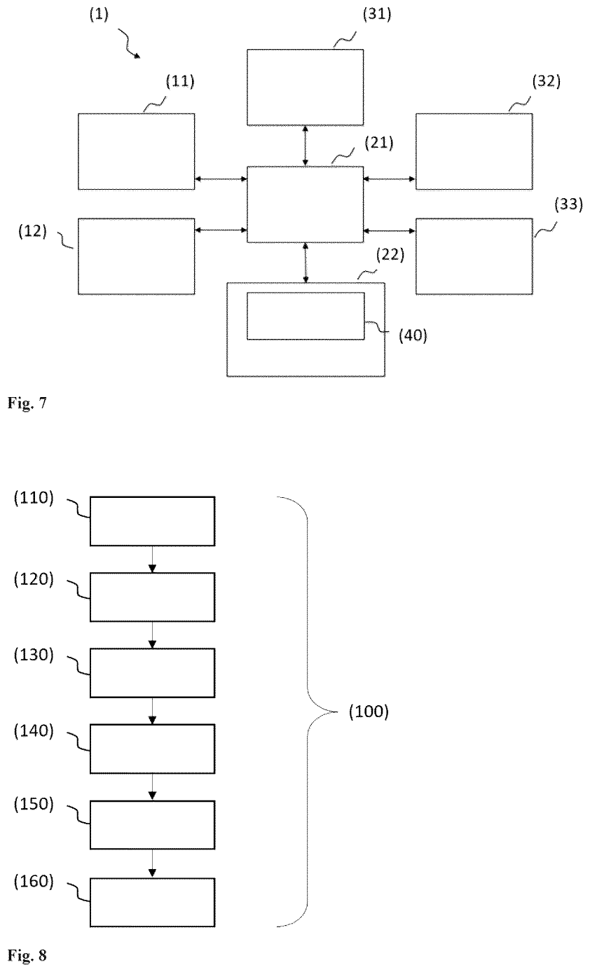

FIG. 7 shows by way of example and in schematic form a further embodiment of the computer system. The computer system (1) comprises a processing unit (21) connected to a storage medium (22). The processing unit (21) and the storage medium (22) form a control and calculation unit, as shown in FIG. 6.

The processing unit (21) may comprise one or more processors alone or in combination with one or more storage media. The processing unit (21) may be customary computer hardware that is able to process information such as digital images, computer programs and/or other digital information. The processing unit (21) normally consists of an arrangement of electronic circuits, some of which can be designed as an integrated circuit or as a plurality of integrated circuits connected to one another (an integrated circuit is sometimes also referred to as a “chip”). The processing unit (21) may be configured to execute computer programs that can be stored in a working memory of the processing unit (21) or in the storage medium (22) of the same or of a different computer system.

The storage medium (22) may be customary computer hardware that is able to store information such as digital images (for example representations of the examination region), data, computer programs and/or other digital information either temporarily and/or permanently. The storage medium (22) may comprise a volatile and/or non-volatile storage medium and may be fixed in place or removable. Examples of suitable storage media are RAM (random access memory), ROM (read-only memory), a hard disk, a flash memory, an exchangeable computer floppy disk, an optical disc, a magnetic tape or a combination of the aforementioned. Optical discs can include compact discs with read-only memory (CD-ROM), compact discs with read/write function (CD-R/W), DVDs, Blu-ray discs and the like.

The processing unit (21) may be connected not just to the storage medium (22), but also to one or more interfaces (11, 12, 31, 32, 33) in order to display, transmit and/or receive information. The interfaces may comprise one or more communication interfaces (11, 32, 33) and/or one or more user interfaces (12, 31). The one or more communication interfaces may be configured to send and/or receive information, for example to and/or from an MRI scanner, a CT scanner, an ultrasound camera, other computer systems, networks, data storage media or the like. The one or more communication interfaces may be configured to transmit and/or receive information via physical (wired) and/or wireless communication connections. The one or more communication interfaces may comprise one or more interfaces for connection to a network, for example using technologies such as mobile telephone, wifi, satellite, cable, DSL, optical fibre and/or the like. In some examples, the one or more communication interfaces may comprise one or more close-range communication interfaces configured to connect devices having close-range communication technologies such as NFC, RFID, Bluetooth, Bluetooth LE, ZigBee, infrared (e.g. IrDA) or the like.

The user interfaces may include a display (31). A display (31) may be configured to display information to a user. Suitable examples thereof are a liquid crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces are a microphone, an image- or video-recording device (for example a camera), a keyboard or a keypad, a joystick, a touch-sensitive surface (separate from a touchscreen or integrated therein) or the like. In some examples, the user interfaces may contain an automatic identification and data capture technology (AIDC) for machine-readable information. This can include barcodes, radiofrequency identification (RFID), magnetic strips, optical character recognition (OCR), integrated circuit cards (ICC) and the like. The user interfaces may in addition comprise one or more interfaces for communication with peripherals such as printers and the like.

One or more computer programs (40) may be stored in the storage medium (22) and executed by the processing unit (21), which is thereby programmed to fulfil the functions described in this description. The retrieving, loading and execution of instructions of the computer program (40) may take place sequentially, such that one instruction at a time is retrieved, loaded and executed. However, the retrieving, loading and/or execution may also take place in parallel.

The computer system of the present disclosure may be designed as a laptop, notebook, netbook and/or tablet PC; it may also be a component of an MRI scanner, a CT scanner or an ultrasound diagnostic device.

The present invention also provides a computer program product. Such a computer program product includes a non-volatile data carrier, for example a CD, a DVD, a USB stick or another data storage medium. A computer program is stored on the data carrier. The computer program can be loaded into a working memory of a computer system (more particularly into a working memory of a computer system of the present disclosure), where it causes the computer system to execute the following steps:
providing a first representation, where the first representation represents an examination region of an examination object without contrast agent or after administration of a first amount of a contrast agent,
providing a second representation, where the second representation represents the examination region of the examination object after administration of a second amount of the contrast agent, the second amount being larger than the first amount,
feeding the first representation and the second representation to a first model, where the first model is configured to generate, based on the first representation and the second representation, a third representation, where the third representation represents the examination region of the examination object after administration of a third amount of the contrast agent, the third amount being different from the first amount and the second amount,
feeding the third representation to a second model,
   where the second model has been trained in a training process based on training data,
   where the training data for each reference object comprise a multiplicity of reference objects:
      (i) a reference representation generated by the first model that represents a reference region of the reference object after administration of a reference amount of the contrast agent, and
      (ii) a measured reference representation of the reference region of the reference object after administration of the reference amount of the contrast agent,
   where the training process for each reference object comprises the steps of
      feeding the reference representation generated by the first model to the second model,
      receiving a corrected reference representation from the second model,
      reducing the differences between the corrected reference representation and the measured reference representation by modifying model parameters,
receiving a corrected third representation of the examination region of the examination object from the second model,
outputting and/or storing the corrected third representation and/or transmitting the corrected third representation to a separate computer system.

The computer program can also be available for purchase as a computer program product as a download, for example via a webpage and/or an app store.

The computer program product can also be marketed in combination (in a set) with the contrast agent. Such a set is also referred to as a kit. Such a kit comprises the contrast agent and the computer program product. It is also possible for such a kit to comprise the contrast agent and means allowing a purchaser to obtain the computer program, for example to download it from a webpage. These means may include a link, i.e. an address of the webpage on which the computer program can be obtained, for example from which the computer program can be downloaded to a computer system connected to the internet. These means can comprise a code (for example an alphanumeric string or a QR code, or a Data Matrix code or a barcode or another optically and/or electronically readable code) that gives the purchaser access to the computer program. Such a link and/or code may for example be printed on a packaging of the contrast agent and/or on a package leaflet of the contrast agent. A kit is thus a combination product comprising a contrast agent and a computer program (e.g. in the form of access to the computer program or in the form of executable program code on a data carrier) that are available for purchase together.

FIG. 8 shows by way of example and in schematic form an embodiment of the computer-implemented method in the form of a flowchart.

The method (100) comprises the steps of:
(110) providing a first representation, where the first representation represents an examination region of an examination object without contrast agent or after administration of a first amount of a contrast agent,
(120) providing a second representation, where the second representation represents the examination region of the examination object after administration of a second amount of the contrast agent, the second amount being larger than the first amount,
(130) feeding the first representation and the second representation to a first model, where the first model is configured to generate, based on the first representation and the second representation, a third representation, where the third representation represents the examination region of the examination object after administration of a third amount of the contrast agent, the third amount being different from the first amount and the second amount,
(140) feeding the third representation to a second model,
   where the second model has been trained in a training process based on training data,
   where the training data for each reference object comprise a multiplicity of reference objects:
      (i) a reference representation generated by the first model that represents a reference region of the reference object after administration of a reference amount of the contrast agent, and
      (ii) a measured reference representation of the reference region of the reference object after administration of the reference amount of the contrast agent,
   where the training process for each reference object comprises the steps of:
      feeding the reference representation generated by the first model to the second model,
      receiving a corrected reference representation from the second model,
      reducing the differences between the corrected reference representation and the measured reference representation by modifying model parameters,
(150) receiving a corrected third representation of the examination region of the examination object from the second model,
(160) outputting and/or storing the corrected third representation and/or transmitting the corrected third representation to a separate computer system.

The present invention can be used for various purposes. Some examples of use are described below, without the invention being limited to these examples of use.

A first example of use concerns magnetic resonance imaging examinations for differentiating intraaxial tumours such as intracerebral metastases and malignant gliomas. The infiltrative growth of these tumours makes it difficult to differentiate exactly between tumour and healthy tissue. Determining the extent of a tumour is however crucial for surgical removal. Distinguishing between tumours and healthy tissue is facilitated by administration of an extracellular; after intravenous administration of a standard dose of 0.1 mmol/kg body weight of the extracellular MRI contrast agent gadobutrol, intraaxial tumours can be differentiated much more readily. At higher doses, the contrast between lesion and healthy brain tissue is increased further; the detection rate of brain metastases increases linearly with the dose of the contrast agent (see for example M. Hartmann et al.: Does the administration of a high dose of a paramagnetic contrast medium (Gadovist) improve the diagnostic value of magnetic resonance tomography in glioblastomas?doi: 10.1055/s-2007-1015623).

A single triple dose or a second subsequent dose may be administered here up to a total dose of 0.3 mmol/kg body weight. This exposes the patient and the environment to additional gadolinium and in the case of a second scan incurs further additional costs.

The present invention can be used to avoid a dose of contrast agent exceeding the standard amount. A first MRI image can be generated without contrast agent or with an amount less than the standard amount and a second MRI image generated with the standard amount. On the basis of these generated MRI images it is possible, as described in this disclosure, to generate a synthetic MRI image in which the contrast between lesions and healthy tissue can be varied within wide limits by altering the gain factor α. This makes it possible to achieve contrasts that are otherwise achievable only by administering an amount of contrast agent larger than the standard amount.

Another example of use concerns the reduction of the amount of MRI contrast agent in a magnetic resonance imaging examination. Gadolinium-containing contrast agents such as gadobutrol are used for a diversity of examinations. They are used for contrast enhancement in examinations of the cranium, spine, chest or other examinations. In the central nervous system, gadobutrol highlights regions where the blood-brain barrier is impaired and/or abnormal vessels. In breast tissue, gadobutrol makes it possible to visualize the presence and extent of malignant breast disease. Gadobutrol is also used in contrast-enhanced magnetic resonance angiography for diagnosing stroke, for detecting tumour blood perfusion and for detecting focal cerebral ischaemia.

Increasing environmental pollution, the cost burden on the health system and the fear of acute side effects and possible long-term health risks, especially in the case of repeated and long-term exposure, have given impetus to efforts to reduce the dose of gadolinium-containing contrast agents. This can be achieved by the present invention.

A first MRI image without contrast agent and a second MRI image with an amount of contrast agent less than the standard amount can be generated. On the basis of these generated MRI images it is possible, as described in this disclosure, to generate a synthetic MRI image in which the contrast can be varied within wide limits by altering the gain factor α. This makes it possible with less than the standard amount of contrast agent to achieve the same contrast as is obtained after administration of the standard amount.

Another example of use concerns the detection, identification and/or characterization of lesions in the liver with the aid of a hepatobiliary contrast agent such as Primovist®.

Primovist® is administered intravenously (i.v.) at a standard dose of 0.025 mmol/kg body weight. This standard dose is lower than the standard dose of 0.1 mmol/kg body weight in the case of extracellular MRI contrast agents. Unlike in contrast-enhanced MRI with extracellular gadolinium-containing contrast agents, Primovist® permits dynamic multiphase T1w imaging. However, the lower dose of Primovist® and the observation of transient motion artefacts that can occur shortly after intravenous administration means that contrast enhancement with Primovist® in the arterial phase is perceived by radiologists as poorer than contrast enhancement with extracellular MRI contrast agents. The assessment of the contrast enhancement in the arterial phase and of the vascularity of focal liver lesions is however of critical importance for accurate characterization of the lesion.

With the aid of the present invention it is possible to increase contrast, particularly in the arterial phase, without the need to administer a higher dose.

A first MRI image without contrast agent and a second MRI image during the arterial phase after administering an amount of a contrast agent that corresponds to the standard amount can be generated. On the basis of these generated MRI images it is possible, as described in this disclosure, to generate a synthetic MRI image in which the contrast in the arterial phase can be varied within wide limits by altering the gain factor α. This makes it possible to achieve contrasts that are otherwise achievable only by administering an amount of contrast agent larger than the standard amount.

Another example of use concerns the use of MRI contrast agents in computed tomography examinations.

In a CT examination, MRI contrast agents usually have a lower contrast-enhancing effect than CT contrast agents. However, it can be advantageous to employ an MRI contrast agent in a CT examination. An example is a minimally invasive intervention in the liver of a patient in whom a surgeon is monitoring the procedure by means of a CT scanner. Computed tomography (CT) has the advantage over magnetic resonance imaging that more major surgical interventions are possible in the examination region while generating CT images of an examination region of an examination object. By contrast, there are only few surgical instruments and surgical devices that are MRI-compatible. Moreover, access to the patient is restricted by the magnets used in MRI. Thus, while performing a procedure in the examination region, a surgeon will be able to visualize the examination region by CT and to follow the procedure on a monitor.

For example, if a surgeon wishes to perform a procedure in a patient's liver in order for example to carry out a biopsy on a liver lesion or to remove a tumour, the contrast between a liver lesion or tumour and healthy liver tissue will not be as pronounced in a CT image of the liver as it is in an MRI image after administration of a hepatobiliary contrast agent. There are currently no known and/or authorized CT-specific hepatobiliary contrast agents in CT. The use of an MRI contrast agent, more particularly a hepatobiliary MRI contrast agent, in computed tomography thus combines the possibility of differentiating between healthy and diseased liver tissue and the possibility of carrying out an operation with simultaneous visualization of the liver.

The comparatively low contrast enhancement achieved by the MRI contrast agent can be increased with the aid of the present invention without the need to administer a dose higher than the standard dose.

A first CT image without MRI contrast agent and a second CT image after administering an amount of an MRI contrast agent that corresponds to the standard amount can be generated. On the basis of these generated CT images it is possible, as described in this disclosure, to generate a synthetic CT image in which the contrast produced by the MRI contrast agent can be varied within wide limits by altering the gain factor α. This makes it possible to achieve contrasts that are otherwise achievable only by administering an amount of MRI contrast agent larger than the standard amount.

1. A computer-implemented method comprising the steps of:
providing a first representation (R1, R1^{I}, R1^{F}), where the first representation (R1, R1^{I}, R1^{F}) represents an examination region of an examination object without contrast agent or after administration of a first amount of a contrast agent,
providing a second representation (R2, R2^{I}, R2^{F}), where the second representation (R2, R2^{I}, R2^{F}) represents the examination region of the examination object after administration of a second amount of the contrast agent, the second amount being larger than the first amount,
feeding the first representation (R1, R1^{I}, R1^{F}) and the second representation (R2, R2^{I}, R2^{F}) to a first model (M1), where the first model (M1) is configured to generate, based on the first representation (R1, R1^{I}, R1^{F}) and the second representation (R2, R2^{I}, R2^{F}), a third representation (R3, R3^{I}, R3^{F}), where the third representation (R3, R3^{I}, R3^{F}) represents the examination region of the examination object after administration of a third amount of the contrast agent, the third amount being different from the first amount and the second amount,
feeding the third representation (R3, R3^{I}, R3^{F}) to a second model (M2),
where the second model (M2) has been trained in a training process based on training data (TD),
where the training data (TD) for each reference object comprises a multiplicity of reference objects:
(i) a reference representation (RR3) generated by the first model that represents a reference region of the reference object after administration of a reference amount of the contrast agent, and
(ii) a measured reference representation (RR3^{M}) of the reference region of the reference object after administration of the reference amount of the contrast agent,
where the training process for each reference object comprises the steps of:
feeding the reference representation generated by the first model (M1) to the second model (M2),
receiving a corrected reference representation (RR3^{C}) from the second model (M2),
reducing the differences between the corrected reference representation (RR3^{C}) and the measured reference representation (RR3^{M}) by modifying model parameters,
receiving a corrected third representation (R3^{C}) of the examination region of the examination object from the second model (M2),
outputting and/or storing the corrected third representation (R3^{C}) and/or transmitting the corrected third representation (R3^{C}) to a separate computer system.

2. The method as claimed in claim 1, wherein the first model (M1) is a mechanistic model.

3. The method as claimed in claim 1, wherein the generation of the third representation (R3, R3^{I}, R3^{F}) by the first model (M1) comprises the steps of:
subtracting the first representation (R1, R^{I}, R1^{F}) from the second representation (R2, R2^{I}, R2^{F}),
multiplying the subtraction result by a gain factor α,
adding the multiplication result to the first representation (R1, R1^{I}, R1^{F}).

4. The method as claimed in claim 1, wherein the generation of the third representation (R1, R1^{I}, R1^{F}) by the first model (M1) comprises the steps of:
multiplying a frequency-space representation (R2^{F}−R1^{F}) of a difference of the first representation (R1, R1^{I}, R1^{F}) from the second representation (R2, R2^{I}, R2^{F}) by a frequency-dependent weight function (WF), thereby obtaining a weighted representation (R2^{F}−R1^{F})^{W},
multiplying the weighted representation (R2^{F}−R1^{F})^{W} by a gain factor α,
adding the weighted representation (R2^{F}−R1^{F})^{W} multiplied by the gain factor α to the first representation (R1, R1^{I}, R1^{F}).

5. The method as claimed in claim 4, wherein the frequency-dependent weight function (WF) is a Hann window function or a Poisson window function.

6. The method as claimed in claim 3, wherein the gain factor α is greater than 1, preferably greater than 2.

7. The method as claimed in claim 3, wherein the gain factor α is greater than zero and less than 1.

8. The method as claimed in claim 3, wherein the gain factor α is less than zero.

9. The method as claimed in claim 1, wherein the third amount and the reference amount are larger than a standard amount of the contrast agent.

10. The method as claimed in claim 1, wherein the second model (M2) is an artificial neural network, wherein the first model (M1) comprises one or more processing layers (L4, L5) prepending the second model (M2).

11. The method as claimed in claim 10, wherein the second model (M2) is prepended by one or more further processing layers (L1, L2, L3), wherein the one or more further processing layers form an initial model (M0), where the initial model (M0) is configured to determine the gain factor α and/or parameters of the frequency-dependent weight function (WF) based on the first representation (R1, R1^{I}, R1^{F}) and/or the second representation (R2, R2^{I}, R2^{F}) and/or to co-register the first representation (R1, R1^{I}, R1^{F}) and the second representation (R2, R2^{I}, R2^{F}).

12. The method as claimed in claim 1, wherein the examination object and each reference object is a human or an animal, preferably a mammal, wherein the examination region is a part of the examination object, where the reference region is a part of the reference object and the reference region corresponds to the examination region.

13. The method as claimed in claim 1, wherein the first representation (R1, R1^{I}, R1^{F}) and the second representation (R2, R2^{I}, R2^{F}) are the result of a radiological examination, preferably an MRI examination and/or a CT examination.

14. The method as claimed in claim 1, wherein the contrast agent comprises
a Gd³⁺ complex of a compound of the formula (I) 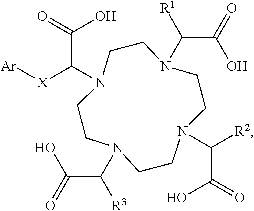
where
Ar is a group selected from 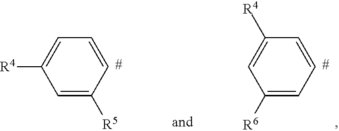
where _{#} is the linkage to X,
X is a group selected from
CH₂, (CH₂)₂, (CH₂)₃, (CH₂)₄ and *—(CH₂)₂O—CH₂—^{#},
where * is the linkage to Ar and ^{#} is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof, or
a Gd³⁺ complex of a compound of the formula (II) 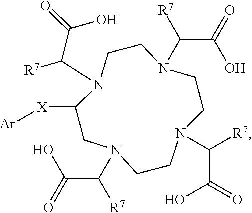
where
Ar is a group selected from 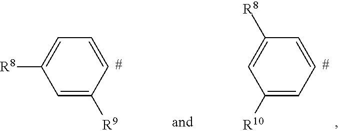
where _{#} is the linkage to X,
X is a group selected from CH₂, (CH₂)₂, (CH₂)₃, (CH₂)₄ and *—(CH₂)₂O—CH₂—^{#}, where * is the linkage to Ar and ^{#} is the linkage to the acetic acid residue,
R⁷ is a hydrogen atom or a group selected from C₁-C₃ alkyl, —CH₂OH,
—(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof, or
the contrast agent comprises one of the following substances:
gadolinium(III) 2-[4,7,10-tris(carboxymethyl)-1,4,7,10-tetrazacyclododec-1-yl]acetic acid,
gadolinium(III) ethoxybenzyldiethylenetriaminepentaacetic acid,
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate,
dihydrogen [(±)-4-carboxy-5,8,11-tris(carboxymethyl)-1-phenyl-2-oxa-5,8,11-triazatridecan-13-oato(5-)]gadolinate(2-),
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]-amino}methyl)-4,7,11,14-tetraazahepta-decan-2-yl}-1,4,7,10-tetraazacyclododecan-1-yl]acetate,
2,2′,2″-(10-{1-carboxy-2-[2-(4-ethoxyphenyl)ethoxy]ethyl}-1,4,7,10-tetraazacyclododecane-1,4,7-triyl)triacetate,
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate,
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate,
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate)
gadolinium 2,2′,2″-{10-[(1S)-4-(4-butoxyphenyl)-1-carboxybutyl]-1,4,7,10-tetraazacyclododecane-1,4,7-triyl}triacetate,
gadolinium(III) 5,8-bis(carboxylatomethyl)-2-[2-(methylamino)-2-oxoethyl]-10-oxo-2,5,8,11-tetraazadodecane-1-carboxylate hydrate
gadolinium(III) 2-[4-(2-hydroxypropyl)-7,10-bis(2-oxido-2-oxoethyl)-1,4,7,10-tetrazacyclododec-1-yl]acetate,
gadolinium(III) 2,2′,2″-(10-((2R,3S)-1,3,4-trihydroxybutan-2-yl)-1,4,7,10-tetraazacyclododecane-1,4,7-triyl)triacetate,
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate,
gadolinium-2,2′,2″-[10-(carboxymethyl)-2-(4-ethoxybenzyl)-1,4,7,10-tetraazacyclododecane-1,4,7-triyl]triacetate.

15. A computer system (1) comprising
a receiving unit (11),
a control and calculation unit (12) and
an output unit (13),
wherein the control and calculation unit (12) is configured
to generate a first representation (R1, R1^{I}, R1^{F}) or cause the receiving unit (11) to receive the first representation (R1, R1^{I}, R1^{F}), where the first representation (R1, R1^{I}, R1^{F}) represents an examination region of an examination object without contrast agent or after administration of a first amount of a contrast agent,
to generate a second representation (R2, R2^{I}, R2^{F}) or to cause the receiving unit (11) to receive the second representation (R2, R2^{I}, R2^{F}), where the second representation (R2, R2^{I}, R2^{F}) represents the examination region of the examination object after administration of a second amount of the contrast agent, the second amount being larger than the first amount,
to feed the first representation (R1, R1^{I}, R1^{F}) and the second representation (R2, R2^{I}, R2^{F}) to a first model (M1), where the first model (M1) is configured to generate, based on the first representation (R1, R1^{I}, R1^{F}) and the second representation (R2, R2^{I}, R2^{F}), a third representation (R3, R3^{I}, R3^{F}), where the third representation (R3, R3^{I}, R3^{F}) represents the examination region of the examination object after administration of a third amount of the contrast agent, the third amount being different from the first amount and the second amount,
to feed the third representation (R3, R3^{I}, R3^{F}) to a second model (M2),
where the second model (M2) has been trained in a training process based on training data (TD),
where the training data (TD) for each reference object comprises a multiplicity of reference objects:
(i) a reference representation (RR3) generated by the first model (M1) that represents a reference region of the reference object after administration of a reference amount of the contrast agent, and
(ii) a measured reference representation (RR3^{M}) of the reference region of the reference object after administration of the reference amount of the contrast agent,
where the training process for each reference object comprises the steps of:
feeding the reference representation (RR3) generated by the first model (M1) to the second model (M2),
receiving a corrected reference representation (RR3^{C}) from the second model (M2),
reducing the differences between the corrected reference representation (RR3^{C}) and the measured reference representation (RR3^{M}) by modifying model parameters (MP),
to receive a corrected third representation (R3c) of the examination region of the examination object from the second model (M2),
to cause the output unit (13) to output the corrected third representation (R3c), store it and/or transmit it to a separate computer system.

16. A computer program product comprising a data carrier on which is stored a computer program (40) that can be loaded into a working memory (22) of a computer system (1), where it causes the computer system (1) to execute the following steps:
providing a first representation (R1, R1^{I}, R1^{F}), where the first representation (R1, R1^{I}, R1^{F}) represents an examination region of an examination object without contrast agent or after administration of a first amount of a contrast agent,
providing a second representation (R2, R2^{I}, R2^{F}), where the second representation (R2, R2^{I}, R2^{F}) represents the examination region of the examination object after administration of a second amount of the contrast agent, the second amount being larger than the first amount,
feeding the first representation (R1, R1^{I}, R1^{F}) and the second representation (R2, R2^{I}, R2^{F}) to a first model (M1), where the first model (M1) is configured to generate, based on the first representation (R1, R1^{I}, R1^{F}) and the second representation (R2, R2^{I}, R2^{F}), a third representation (R3, R3^{I}, R3^{F}), where the third representation (R3, R3^{I}, R3^{F}) represents the examination region of the examination object after administration of a third amount of the contrast agent, the third amount being different from the first amount and the second amount,
feeding the third representation (R3, R3^{I}, R3^{F}) to a second model (M2),
where the second model (M2) has been trained in a training process based on training data (TD),
where the training data (TD) for each reference object comprises a multiplicity of reference objects:
(i) a reference representation (RR3) generated by the first model that represents a reference region of the reference object after administration of a reference amount of the contrast agent, and
(ii) a measured reference representation (RR3^{M}) of the reference region of the reference object after administration of the reference amount of the contrast agent,
where the training process for each reference object comprises the steps of:
feeding the reference representation (RR3) generated by the first model (M1) to the second model (M2),
receiving a corrected reference representation (RR3^{C}) from the second model (M2),
reducing the differences between the corrected reference representation (RR3^{C}) and the measured reference representation (RR3^{M}) by modifying model parameters (MP),
receiving a corrected third representation (R3^{C}) of the examination region of the examination object from the second model (M2),
outputting and/or storing the corrected third representation (R3^{C}) and/or transmitting the corrected third representation (R3^{C}) to a separate computer system.

17. The use of a contrast agent in a radiological examination method comprising the steps of:
providing a first representation (R1, R1^{I}, R1^{F}), where the first representation (R1, R1^{I}, R1^{F}) represents an examination region of an examination object without the contrast agent or after administration of a first amount of the contrast agent,
providing a second representation (R2, R2^{I}, R2^{F}), where the second representation (R2, R2^{I}, R2^{F}) represents the examination region of the examination object after administration of a second amount of the contrast agent, the second amount being larger than the first amount,
feeding the first representation (R1, R1^{I}, R1^{F}) and the second representation (R2, R2^{I}, R2^{F}) to a first model (M1), where the first model (M1) is configured to generate, based on the first representation (R1, R1^{I}, R1^{F}) and the second representation (R2, R2^{I}, R2^{F}), a third representation (R3, R3^{I}, R3^{F}), where the third representation (R3, R3^{I}, R3^{F}) represents the examination region of the examination object after administration of a third amount of the contrast agent, the third amount being different from the first amount and the second amount,
feeding the third representation (R3, R3^{I}, R3^{F}) to a second model (M2),
where the second model (M2) has been trained in a training process based on training data (TD),
where the training data (TD) for each reference object comprises a multiplicity of reference objects:
(i) a reference representation (RR3) generated by the first model that represents a reference region of the reference object after administration of a reference amount of the contrast agent, and
(ii) a measured reference representation (RR3^{M}) of the reference region of the reference object after administration of the reference amount of the contrast agent,
where the training process for each reference object comprises the steps of:
feeding the reference representation (RR3) generated by the first model (M1) to the second model (M2),
receiving a corrected reference representation (RR3^{C}) from the second model (M2),
reducing the differences between the corrected reference representation (RR3^{C}) and the measured reference representation (RR3^{M}) by modifying model parameters (MP),
receiving a corrected third representation (R3^{C}) of the examination region of the examination object from the second model (M2),
outputting and/or storing the corrected third representation (R3^{C}) and/or transmitting the corrected third representation (R3^{C}) to a separate computer system.

18. The use as claimed in claim 17, wherein the radiological examination method is a magnetic resonance imaging examination or a computed tomography examination and wherein the contrast agent comprises
a Gd³⁺ complex of a compound of the formula (I) 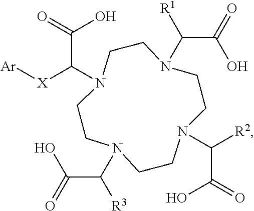
where
Ar is a group selected from 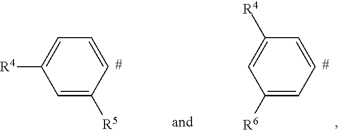
where _{#} is the linkage to X,
X is a group selected from
CH₂, (CH₂)₂, (CH₂)₃, (CH₂)₄ and *—(CH₂)₂O—CH₂—^{#},
where * is the linkage to Ar and ^{#} is the linkage to the acetic acid residue,
R1, R2 and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof, or
a Gd³⁺ complex of a compound of the formula (II) 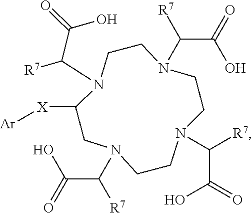
where
Ar is a group selected from 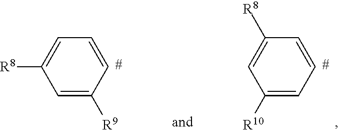
where _{#} is the linkage to X,
X is a group selected from CH₂, (CH₂)₂, (CH₂)₃, (CH₂)₄ and *—(CH₂)₂O—CH₂—^{#}, where * is the linkage to Ar and ^{#} is the linkage to the acetic acid residue,
R⁷ is a hydrogen atom or a group selected from C₁-C₃ alkyl, —CH₂OH, —(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof, or
the contrast agent comprises one of the following substances:
gadolinium(III) 2-[4,7,10-tris(carboxymethyl)-1,4,7,10-tetrazacyclododec-1-yl]acetic acid,
gadolinium(III) ethoxybenzyldiethylenetriaminepentaacetic acid,
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate,
dihydrogen [(±)-4-carboxy-5,8,11-tris(carboxymethyl)-1-phenyl-2-oxa-5,8,11-triazatridecan-13-oato(5-)]gadolinate(2-),
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]-amino}methyl)-4,7,11,14-tetraazahepta-decan-2-yl}-1,4,7,10-tetraazacyclododecan-1-yl]acetate,
2,2′,2″-(10-{1-carboxy-2-[2-(4-ethoxyphenyl)ethoxy]ethyl}-1,4,7,10-tetraazacyclododecane-1,4,7-triyl)triacetate,
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate,
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate,
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate)
gadolinium 2,2′,2″-{10-[(1S)-4-(4-butoxyphenyl)-1-carboxybutyl]-1,4,7,10-tetraazacyclododecane-1,4,7-triyl}triacetate,
gadolinium(III) 5,8-bis(carboxylatomethyl)-2-[2-(methylamino)-2-oxoethyl]-10-oxo-2,5,8,11-tetraazadodecane-1-carboxylate hydrate
gadolinium(III) 2-[4-(2-hydroxypropyl)-7,10-bis(2-oxido-2-oxoethyl)-1,4,7,10-tetrazacyclododec-1-yl]acetate,
gadolinium(III) 2,2′,2″-(10-((2R,3S)-1,3,4-trihydroxybutan-2-yl)-1,4,7,10-tetraazacyclododecane-1,4,7-triyl)triacetate,
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate,
gadolinium-2,2′,2″-[10-(carboxymethyl)-2-(4-ethoxybenzyl)-1,4,7,10-tetraazacyclododecane-1,4,7-triyl]triacetate.

19. A contrast agent for use in a radiological examination method comprising the steps of:
providing a first representation (R1, R1^{I}, R1^{F}), where the first representation (R1, R1^{I}, RIF) represents an examination region of an examination object without the contrast agent or after administration of a first amount of the contrast agent,
providing a second representation (R2, R2^{I}, R2^{F}), where the second representation (R2, R2^{I}, R2^{F}) represents the examination region of the examination object after administration of a second amount of the contrast agent, the second amount being larger than the first amount,
feeding the first representation (R1, R1^{I}, R1^{F}) and the second representation (R2, R2^{I}, R2^{F}) to a first model (M1), where the first model (M1) is configured to generate, based on the first representation (R1, R1^{I}, R1^{F}) and the second representation (R2, R2^{I}, R2^{F}), a third representation (R3, R3^{I}, R3^{F}), where the third representation (R3, R3^{I}, R3^{F}) represents the examination region of the examination object after administration of a third amount of the contrast agent, the third amount being different from the first amount and the second amount,
feeding the third representation (R3, R3^{I}, R3^{F}) to a second model (M2),
where the second model (M2) has been trained in a training process based on training data (TD),
where the training data (TD) for each reference object comprises a multiplicity of reference objects:
(i) a reference representation (RR3) generated by the first model that represents a reference region of the reference object after administration of a reference amount of the contrast agent, and
(ii) a measured reference representation (RR3^{M}) of the reference region of the reference object after administration of the reference amount of the contrast agent,
where the training process for each reference object comprises the steps of:
feeding the reference representation (RR3) generated by the first model (M1) to the second model (M2),
receiving a corrected reference representation (RR3^{C}) from the second model (M2),
reducing the differences between the corrected reference representation (RR3^{C}) and the measured reference representation (RR3^{M}) by modifying model parameters (MP),
receiving a corrected third representation (R3^{C}) of the examination region of the examination object from the second model (M2),
outputting and/or storing the corrected third representation (R3^{C}) and/or transmitting the corrected third representation (R3^{C}) to a separate computer system.

20. The contrast agent for use as claimed in claim 18, wherein the radiological examination method is a magnetic resonance imaging examination or a computed tomography examination and wherein the contrast agent comprises
a Gd³⁺ complex of a compound of the formula (I) 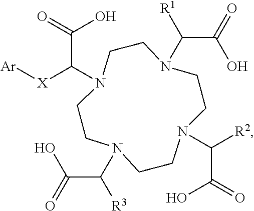
where
Ar is a group selected from 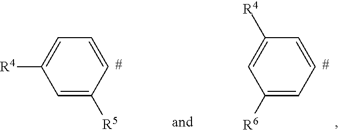
where _{#} is the linkage to X,
X is a group selected from
CH₂, (CH₂)₂, (CH₂)₃, (CH₂)₄ and *—(CH₂)₂O—CH₂—^{#},
where * is the linkage to Ar and ^{#} is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof, or
a Gd³⁺ complex of a compound of the formula (II) 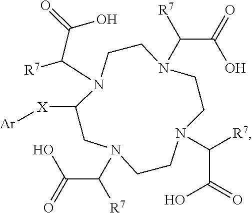
where
Ar is a group selected from 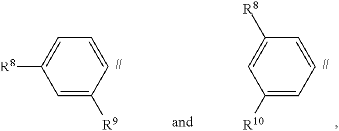
where _{#} is the linkage to X,
X is a group selected from CH₂, (CH₂)₂, (CH₂)₃, (CH₂)₄ and *—(CH₂)₂O—CH₂—^{#}, where * is the linkage to Ar and ^{#} is the linkage to the acetic acid residue,
R⁷ is a hydrogen atom or a group selected from C₁-C₃ alkyl, —CH₂OH, —(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof, or
the contrast agent comprises one of the following substances:
gadolinium(III) 2-[4,7,10-tris(carboxymethyl)-1,4,7,10-tetrazacyclododec-1-yl]acetic acid,
gadolinium(III) ethoxybenzyldiethylenetriaminepentaacetic acid,
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate,
dihydrogen [(±)-4-carboxy-5,8,11-tris(carboxymethyl)-1-phenyl-2-oxa-5,8,11-triazatridecan-13-oato(5-)]gadolinate(2-),
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]-amino}methyl)-4,7,11,14-tetraazahepta-decan-2-yl}-1,4,7,10-tetraazacyclododecan-1-yl]acetate,
2,2′,2″-(10-{1-carboxy-2-[2-(4-ethoxyphenyl)ethoxy]ethyl}-1,4,7,10-tetraazacyclododecane-1,4,7-triyl)triacetate,
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate,
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate,
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate)
gadolinium 2,2′,2″-{10-[(1S)-4-(4-butoxyphenyl)-1-carboxybutyl]-1,4,7,10-tetraazacyclododecane-1,4,7-triyl}triacetate,
gadolinium(III) 5,8-bis(carboxylatomethyl)-2-[2-(methylamino)-2-oxoethyl]-10-oxo-2,5,8,11-tetraazadodecane-1-carboxylate hydrate
gadolinium(III) 2-[4-(2-hydroxypropyl)-7,10-bis(2-oxido-2-oxoethyl)-1,4,7,10-tetrazacyclododec-1-yl]acetate,
gadolinium(III) 2,2′,2″-(10-((2R,3S)-1,3,4-trihydroxybutan-2-yl)-1,4,7,10-tetraazacyclododecane-1,4,7-triyl)triacetate,
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate,
gadolinium-2,2′,2″-[10-(carboxymethyl)-2-(4-ethoxybenzyl)-1,4,7,10-tetraazacyclododecane-1,4,7-triyl]triacetate.

21. A kit comprising a computer program product as claimed in claim 16 and a contrast agent, wherein the contrast agent preferably comprises
a Gd³⁺ complex of a compound of the formula (I) 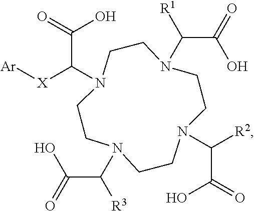
where
Ar is a group selected from 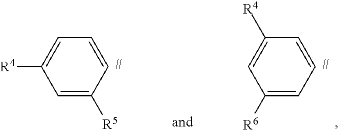
where _{#} is the linkage to X,
X is a group selected from
CH₂, (CH₂)₂, (CH₂)₃, (CH₂)₄ and *—(CH₂)₂O—CH₂—^{#},
where * is the linkage to Ar and ^{#} is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof, or
a Gd³⁺ complex of a compound of the formula (II) 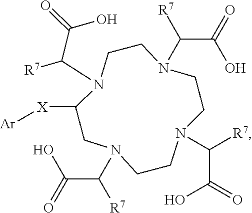
where
Ar is a group selected from 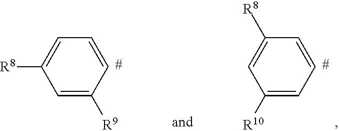
where _{#} is the linkage to X,
X is a group selected from CH₂, (CH₂)₂, (CH₂)₃, (CH₂)₄ and *—(CH₂)₂O—CH₂—^{#}, where * is the linkage to Ar and ^{#} is the linkage to the acetic acid residue,
R⁷ is a hydrogen atom or a group selected from C₁-C₃ alkyl, —CH₂OH, —(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof, or
the contrast agent comprises one of the following substances:
gadolinium(III) 2-[4,7,10-tris(carboxymethyl)-1,4,7,10-tetrazacyclododec-1-yl]acetic acid,
gadolinium(III) ethoxybenzyldiethylenetriaminepentaacetic acid,
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate,
dihydrogen [(±)-4-carboxy-5,8,11-tris(carboxymethyl)-1-phenyl-2-oxa-5,8,11-triazatridecan-13-oato(5-)]gadolinate(2-),
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]-amino}methyl)-4,7,11,14-tetraazahepta-decan-2-yl}-1,4,7,10-tetraazacyclododecan-1-yl]acetate,
2,2′,2″-(10-{1-carboxy-2-[2-(4-ethoxyphenyl)ethoxy]ethyl}-1,4,7,10-tetraazacyclododecane-1,4,7-triyl)triacetate,
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate,
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate,
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate)
gadolinium 2,2′,2″-{10-[(1S)-4-(4-butoxyphenyl)-1-carboxybutyl]-1,4,7,10-tetraazacyclododecane-1,4,7-triyl}triacetate,
gadolinium(III) 5,8-bis(carboxylatomethyl)-2-[2-(methylamino)-2-oxoethyl]-10-oxo-2,5,8,11-tetraazadodecane-1-carboxylate hydrate
gadolinium(III) 2-[4-(2-hydroxypropyl)-7,10-bis(2-oxido-2-oxoethyl)-1,4,7,10-tetrazacyclododec-1-yl]acetate,
gadolinium(III) 2,2′,2″-(10-((2R,3S)-1,3,4-trihydroxybutan-2-yl)-1,4,7,10-tetraazacyclododecane-1,4,7-triyl)triacetate,
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate,
gadolinium-2,2′,2″-[10-(carboxymethyl)-2-(4-ethoxybenzyl)-1,4,7,10-tetraazacyclododecane-1,4,7-triyl]triacetate.
